(19) United States
(12) Patent Application Publication
(10) Pub. No.: US 2024/0219289 A1
(43) Pub. Date: Jul. 4, 2024

(54) DEVICES AND METHODS FOR LABEL-FREE SENSING OF LYMPHOCYTE ACTIVATION AND IDENTITY

(71) Applicant: Wisconsin Alumni Research Foundation, Madison, WI (US)

(72) Inventors: Melissa C. Skala, Middleton, WI (US); Rebecca Schmitz, Madison, WI (US); Alexandra Jule Walsh, Madison, WI (US)

(21) Appl. No.: 18/397,844

(22) Filed: Dec. 27, 2023

Related U.S. Application Data
(60) Provisional application No. 63/437,101, filed on Jan. 4, 2023.

(51) Int. Cl. G01N 15/149; A61K 35/17; G01N 15/1429

(52) U.S. Cl. CPC G01N 15/149; A61K 35/17; G01N 15/1429

(57) ABSTRACT

Devices and methods for label-free sensing of lymphocyte activation and identify are disclosed. The activation status of B cells and NK cells can be reliably determined. A general classifier capable of determining the activation status of lymphocytes having unknown identity (i.e., unknown whether a T cell, B cell, or NK cell) is disclosed. An identity classifier capable of differentiating T cells from B cells from NK cells is disclosed. A six-class classifier is disclosed which is capable of identifying both lymphocyte identity and activation status.

### CROSS-REFERENCE TO RELATED APPLICATIONS

This application is related to, claims priority to, and incorporates herein by reference for all purposes U.S. Provisional Patent Application No. 63/437,101, filed Jan. 4, 2023.

### STATEMENT REGARDING FEDERALLY FUNDED RESEARCH

This invention was made with government support under CA205101 awarded by the National Institutes of Health. The government has certain rights in the invention.

### BACKGROUND

Lymphocytes consist of natural killer (NK) cells, B cells, and T cells, and constitute approximately 20-40% of circulating white blood cells. T cells have diverse cytotoxic and immune-modulating activities after activation, and therapies that modulate T cell function are in development or clinical use for a range of diseases, including cancer, HIV, autoimmune disease, and transplant rejection. NK cells are cytotoxic and surveil the body for unhealthy cells. However, NK cells are not antigen-specific, and instead rely on a balance of activating and inhibitory signals to initiate cytotoxicity. Their cytotoxic function has led to particular interest in the role of NK cells in tumor cell clearance and in adoptive cell therapy for cancer. B cells, like T cells, are a part of the adaptive immune system, and their primary role is the production of antibodies. B cells are also antigen-presenting cells that can present peptides to T cells to promote their effector functions. Subtypes of B cells also secrete cytokines that can either attenuate or suppress the function of surrounding immune cells. The multiple functions of B cells provide several avenues for leveraging B cells as a platform for cell-based therapies, including antigen-presenting B cells as a cancer immunotherapy and protein production for rare genetic diseases. Immune profiling measures activation of NK, B, and T cells to a stimulus (such as an antigen from a virus or bacterium), which can be used to identify individual response and potentially predict outcome. Therefore, the presence and activation of lymphocytes is important for monitoring immune health and response to therapy across a range of diseases.

Given the relevance of NK cells, B cells, and T cells for cell therapy, immunotherapy, infectious disease, and immune profiling, new label-free and non-destructive tools are needed to assess lymphocyte activation and subtype in single cells. Current methods include flow cytometry, cytokine release, single-cell RNA sequencing, and cytometry by time of flight (CyTOF). Flow cytometry provides single-cell resolution, but requires labelling with fluorescent antibodies that can be time consuming and may be disruptive to cells.¹² Bulk measurements of cytokine release are also popular but do not provide single-cell measurements, and ELISPOT, which provides single-cell cytokine release information requires cell labeling.¹² Additionally, cytokine based techniques cannot provide information about subsets of immune cells that do not secrete cytokines. Finally, single-cell RNA sequencing and CyTOF provide extensive single-cell information, but destroy the sample.

### SUMMARY

In an aspect, the present disclosure provides a lymphocyte activation and/or identification status sensing device. The device includes a cell analysis observation zone, an autofluorescence spectrometer, a processor, and a non-transitory computer-readable medium. The cell analysis observation zone is adapted to receive a lymphocyte and to present the lymphocyte for individual autofluorescence interrogation. The autofluorescence spectrometer is configured to acquire an autofluorescence data set for the lymphocyte located in the cell analysis observation zone. The autofluorescence spectrometer includes a light source, a photon-counting detector, and photon-counting electronics. The processor is in electronic communication with the autofluorescence spectrometer. The non-transitory computer-readable medium is accessible to the processor and has stored thereon instructions that, when executed by the processor, cause the processor to: a) receive the autofluorescence data set; and b) identify a current activation and/or identification status of the lymphocyte based on a current activation and/or identification prediction. The current activation and/or identification prediction is computed using at least a portion of the autofluorescence data set. The current activation and/or identification prediction can be: i) a six-class classification; ii) a lymphocyte identification prediction; iii) a lymphocyte activation prediction; iv) a B cell activation prediction; or v) a NK cell activation prediction.

In another aspect, methods of classification are disclosed using the acquired autofluorescence data set and the above-referenced classification and predictions.

The six-class classification predicts that the lymphocyte is an activated T cell, a quiescent T cell, an activated B cell, a quiescent B cell, an activated NK cell, or a quiescent NK cell. The six-class classification is computed using at least three six-class metabolic endpoints. The at least three six-class metabolic endpoints include reduced nicotinamide adenine dinucleotide and/or reduced nicotinamide dinucleotide phosphate (NAD(P)H) shortest fluorescence amplitude component (α₁), NAD(P)H shortest fluorescence lifetime component (τ₁), and NAD(P)H mean fluorescence lifetime (τₘ).

The lymphocyte identification prediction predicts that the lymphocyte is a T cell, a B ell, or a NK cell. The lymphocyte identification prediction is computed using at least two metabolic endpoints as an input. The at least two lymphocyte identification metabolic endpoints include flavin adenine dinucleotide (FAD) τₘ, FAD τ₁, or NAD(P)H τₘ.

The lymphocyte activation prediction predicts that the lymphocyte is an activated lymphocyte or a quiescent lymphocyte. The lymphocyte activation prediction is computed using at least two lymphocyte activation metabolic endpoints of the autofluorescence data set as an input. The at least two lymphocyte activation metabolic endpoints include NAD(P)H α₁ and one of an optical redox ratio, NAD(P)H τ₁, or NAD(P)H τₘ.

The B cell activation predicts that the lymphocyte (in most cases, known in advance to be a B cell) is an activated B cell or a quiescent B cell. The B cell activation prediction is computed using at least two B cell activation metabolic endpoints as an input. The at least two B cell metabolic endpoints include NAD(P)H α₁ and NAD(P)H τₘ.

The NK cell activation prediction predicts that the lymphocyte (in most cases, known in advance to be a NK cell) is an activate NK cell or a quiescent NK cell. The NK cell activation prediction is computed using at least two NK cell activation metabolic endpoints as an input. The at least two NK activation metabolic endpoints include either: NAD(P)H α₁ and an optical redox ratio; or NAD(P)H α₁, NAD(P)H τₘ, and NAD(P)H second shortest lifetime (τ₂), and NAD(P)H τ₁.

### BRIEF DESCRIPTIONS OF THE DRAWINGS AND APPENDIX

FIG. 1 is a flowchart illustrating a method, in accordance with an aspect of the present disclosure.

FIG. 2 is a flowchart illustrating a method, in accordance with an aspect of the present disclosure.

FIG. 3 is a flowchart illustrating a method, in accordance with an aspect of the present disclosure.

FIG. 4 is a block diagram of a device, in accordance with an aspect of the present disclosure.

FIG. 5 shows optical metabolic imaging of primary human B cells activated with IL-4 and anti-CD40. (A) B cells were isolated from human peripheral blood of three different donors and activated for 72 hours with 5 μg/mL anti-CD40 and 20 ng/ml IL-4, or cultured unstimulated. (B) IL-6 concentration was measured in media collected from B cells isolated from two different donors and cultured with or without anti-CD40/IL-4 for 72 hours. The increase in IL-6 concentration in the activated B cell condition is consistent with T-cell dependent B cell activation. **** P<0.0001, parametric T-test. (C) Samples of media from activated and quiescent B cells were taken before imaging and measured using commercial kits. Glucose in the media of activated B cells was significantly decreased compared to the quiescent cell media. (D) Lactate levels in activated B cell media were significantly higher than lactate levels in the quiescent cell media. (E) Representative images of NAD(P)H τₘ, FAD τₘ, redox ratio (NAD(P)H intensity divided by the sum of NAD(P)H and FAD intensity), and anti-CD69 staining in the unstimulated and activated conditions. (F) Redox ratio normalized to the mean of the control group significantly increased in the CD69+ B cells in the IL-4+anti-CD40 condition compared to CD69− B cells in the unstimulated condition. (G)-(H) NAD(P)H τₘ significantly decreased and NAD(P)H α₁ significantly increased in the CD69+ B cells in the IL-4+ anti-CD40 condition compared to CD69− B cells in the unstimulated condition. (I) A significant decrease in FAD τₘ was seen in the CD69+ B cells in the IL-4+anti-CD40 condition compared to CD69− B cells in the unstimulated condition. In (C)-(D), media samples were diluted 100-fold and 0.5 μL was assayed. Assays were performed according to the respective BioVision kit protocols. * P<0.05, **** P<0.0001, parametric T-test. In (F)-(I), data are displayed as box-and-whisker plots, representing the median and interquartile range (IQR), with whiskers at 1.5*IQR. Plots are overlaid with data points; each point represents one cell. n=1210 cells (461 cells in the activated CD69+ condition, 749 cells in the control CD69− condition). **** P<0.0001, two-tailed unpaired T-test.

FIG. 6 shows heterogeneity and classification of activated and quiescent B cells using OMI parameters. (A) B cells were isolated from human peripheral blood of three different donors and activated for 72 hours with 5 μg/mL anti-CD40 and 20 ng/ml IL-4, or cultured unstimulated. (B) IL-6 concentration was measured in media collected from B cells isolated from two different donors and cultured with or without anti-CD40/IL-4 for 72 hours. The increase in IL-6 concentration in the activated B cell condition is consistent with T-cell dependent B cell activation. **** P<0.0001, parametric T-test. (C) Samples of media from activated and quiescent B cells were taken before imaging and measured using commercial kits. Glucose in the media of activated B cells was significantly decreased compared to the quiescent cell media. (D) Lactate levels in activated B cell media were significantly higher than lactate levels in the quiescent cell media. (E) Representative images of NAD(P)H τₘ, FAD τₘ, redox ratio (NAD(P)H intensity divided by the sum of NAD(P)H and FAD intensity), and anti-CD69 staining in the unstimulated and activated conditions. (F) Redox ratio normalized to the mean of the control group significantly increased in the CD69+ B cells in the IL-4+anti-CD40 condition compared to CD69− B cells in the unstimulated condition. (G)-(H) NAD(P)H τₘ significantly decreased and NAD(P)H α₁ significantly increased in the CD69+ B cells in the IL-4+anti-CD40 condition compared to CD69− B cells in the unstimulated condition. (I) A significant decrease in FAD τₘ was seen in the CD69+ B cells in the IL-4+anti-CD40 condition compared to CD69− B cells in the unstimulated condition. In (C)-(D), media samples were diluted 100-fold and 0.5 μL was assayed. Assays were performed according to the respective BioVision kit protocols. * P<0.05, **** P<0.0001, parametric T-test. In (F)-(I), data are displayed as box-and-whisker plots, representing the median and interquartile range (IQR), with whiskers at 1.5*IQR. Plots are overlaid with data points; each point represents one cell. n=1210 cells (461 cells in the activated CD69+ condition, 749 cells in the control CD69− condition). **** P<0.0001, two-tailed unpaired T-test.

FIG. 7 shows optical metabolic imaging of primary human NK cells activated with IL-12, IL-15, and IL-18. (A) NK cells were isolated from human peripheral blood of three different donors and activated with 10 ng/ml IL-12, 50 ng/ml IL-15, and 50 ng/mL IL-18 for 24 hours. (B) IFN-γ concentration in media collected from NK-cells isolated from two different donors and cultured with or without activating cytokines for 24 hours. The increase of IFN-γ in the activated condition is consistent with NK cell activation. **** P<0.0001, parametric T-test. (C) Samples of media from activated and quiescent NK cells from two different donors were taken before imaging and measured using commercial kits. Glucose in the media of activated NK cells was significantly decreased compared to the quiescent cell media. (D) Lactate levels in activated NK cell media were significantly higher than lactate levels in the quiescent cell media. (E) Representative images of NAD(P)H τₘ, FAD τₘ, redox ratio, and anti-CD69 staining in the control and activated conditions. (F) Redox ratio significantly increased in the CD69+ NK cells in the cytokine-activated condition compared to CD69− NK cells in the unstimulated condition. (G)-(I) NAD(P)H τₘ significantly decreased, and FAD τₘ and NAD(P)H α₁ significantly increased, in the CD69+ NK cells in the cytokine-activated condition compared to CD69− NK cells in the unstimulated condition. In (C)-(D), media samples were diluted 100-fold and 0.5 μL was assayed. Assays were performed according to the respective BioVision kit protocols. *** P<0.001, **** P<0.0001, parametric T-test. In (F)-(I), data are displayed as box-and-whisker plots, representing the median and interquartile range (IQR), with whiskers at 1.5*IQR. Plots are overlaid with data points; each point represents one cell. n=1221 cells (554 cells in the activated CD69+ condition, 667 cells in the control CD69− condition). **** P<0.0001, two-tailed unpaired T-test.

FIG. 8 shows heterogeneity and classification of activated and quiescent NK cells using OMI parameters. (A) Heatmap of single-cell data across all NK cell experiments reveals heterogeneity within the dataset. Hierarchical cell clustering was calculated on the z-scores (the difference between cell mean and population mean divided by the population standard deviation) of nine OMI variables (NAD(P)H τₘ, τ₁, τ₂, α₁; FAD τₘ, τ₁, τ₂, α₁; and control-normalized optical redox ratio). (B) UMAP of nine OMI parameters displays clustering of activated (CD69+ in activated condition) and quiescent (CD69− in unstimulated condition) NK cells. (C) Pie chart showing the relative weight of each of the nine OMI parameters in the “all variable” random forest classifier. (D) ROC curve of random forest classifiers trained for classification of quiescent and activated NK cells based on different combinations of OMI parameters, with operating points indicated. “Top variables” classifiers refer to the largest weighted OMI parameters in the classifier using all variables, displayed in (C). The classifier using the top four OMI parameters performed the best (AUC 0.97), followed by the classifier that used all 9 OMI parameters (AUC 0.96) and the classifier that used only NAD(P)H lifetime variables (NAD(P)H τₘ, τ₁, τ₂, α₁) (AUC 0.96). n=1221 cells (554 cells in the activated CD69+ condition, 667 cells in the control CD69− condition) with a 70/30 split for training and test sets.

FIG. 9 shows classification of lymphocyte activation status based on OMI parameters collected in B cells, NK cells, and T-cells. Data from activated and quiescent T cells, B cells, and NK cells was used to evaluate OMI measurements across lymphocytes. T cell data where T cells were activated with CD2/3/28 for 48 h and imaged with OMI. (A) Box-and-whisker plots of key OMI variables (control-normalized optical redox ratio, NAD(P)H τₘ, and NAD(P)H α₁) display consistent changes with activation across T cells, B cells, and NK cells. Additional changes were noted between quiescent (CD69− control) cells in each of the three lymphocyte subtypes (comparisons between quiescent groups were interpreted as not meaningful for the optical redox ratio, due to normalization). (B) Heatmap displaying hierarchical clustering of groups of activated or quiescent cells by lymphocyte subtype, donor, and activation status, calculated from the z-scores (the difference between experimental group mean and the mean of all cells divided by the standard deviation of all cells) of nine OMI variables. (C) UMAP of single-cell OMI data displays distinct clusters of lymphocytes based on lymphocyte subtype and activation status. (D) ROC curves of random forest classifiers trained to identify activated cells across all three lymphocyte subtypes, with operating points indicated. The highest weighted OMI parameters were used in the “top variables” classifiers; these weights are in FIG. 14. (E) Accuracy of different random forest classifiers trained to identify lymphocyte subtype (one vs one approach). Variable weights for “top variables” are in FIG. 15B. (F) Accuracy of random forest classifiers trained to identify lymphocyte subtype and activation across all three lymphocyte subtypes (one vs. one approach) using different OMI parameters. Variable weights are in FIG. 17B. n=3127 cells (749 CD69− control B cells, 461 CD69+ activated B cells, 667 CD69− control NK cells, 554 CD69+ activated NK cells, 331 CD69− control T cells, 365 CD69+ activated T cells) with a 50/50 split for training and test sets. **** P<0.0001, Kruskal-Wallis with post-hoc comparisons. ns=not significant.

FIG. 10 shows OMI of CD69+ and CD69− B cells in both control and anti-CD40+ IL-4 activated culture. Both CD69+ and CD69− B cells in both conditions (control and IL-4+anti-CD40 activated) for each OMI parameter: (A) optical redox ratio (NAD(P)H intensity divided by the sum of NAD(P)H+FAD intensities), (B) NAD(P)H mean lifetime τₘ, (C) FAD mean lifetime τₘ, (D) unbound NAD(P)H fraction α₁, (E) unbound NAD(P)H lifetime τ₁, (F) protein-bound NAD(P)H lifetime τ₂, (G) protein-bound FAD fraction α₁, (H) protein-bound FAD lifetime τ₁, (I) unbound FAD lifetime τ₂. Plots display single cell values (dots) overlaid on box- and whisker plots displaying the median, interquartile range (IQR), and minimum/maximum value. n=1352 (461 cells in the activated CD69+ condition, 130 cells in the activated CD69− condition, 12 cells in the control CD69+ condition, 749 cells in the control CD69− condition). *** p<0.005, **** p<0.0001, Kruskal-Wallis with post-hoc comparisons. ns=not significant.

FIG. 11 shows additional UMAPs and classifier performance for single B cell OMI. (A) UMAP of B cells with labels for both CD69+ and CD69− cells in the control and activated (anti-CD40+IL4) groups. (B) UMAP of B-cells color-coded by donor (A, B, C) and activation status (CD69+, CD69−). (C) Confusion matrix of the 9 OMI parameter random forest classifier shows performance for classification of CD69+ activated and CD69− control B cells. (D) Confusion matrix of a logistic regression classifier trained on 9 OMI parameters to classify B cells as CD69+ activated or CD69− control. (E) Confusion matrix of a support vector machine (SVM) classifier trained on 9 OMI parameters to classify B cells as CD69+ activated or CD69− control. (F) ROC curves for random forest, logistic regression, and SVM classifiers trained on 9 OMI parameters, with operating points indicated. In (A), n=1352 (461 cells in the activated CD69+ condition, 130 cells in the activated CD69− condition, 12 cells in the control CD69+ condition, 749 cells in the control CD69− condition). In (B)-(F), n=1210 cells (461 cells in the activated CD69+ condition, 749 cells in the control CD69− condition) with a 70/30 split for training and test sets.

FIG. 12 shows OMI of CD69+ and CD69− NK cells in both unstimulated and IL-12+IL-15+IL-18 activated culture conditions. Both CD69+ and CD69− NK cells in both conditions (control and IL-12+IL-15+IL-18 activated) for each OMI parameter: (A) optical redox ratio, (B) NAD(P)H mean lifetime τₘ, (C) FAD mean lifetime τₘ, (D) unbound NAD(P)H fraction α₁, (E) unbound NAD(P)H lifetime τ₁, (F) protein-bound NAD(P)H lifetime τ₂, (G) protein-bound FAD fraction α₁, (H) protein-bound FAD lifetime τ₁, (I) unbound FAD lifetime τ₂. Plots display single cell values (dots) overlaid on box- and whisker plots displaying the median, interquartile range (IQR), and minimum/maximum value. n=1642 cells (554 activated CD69+ cells, 372 activated CD69− cells, 49 control CD69+ cells, 667 control CD69− cells). ** p<0.01, *** p<0.001, **** p<0.0001, Kruskal-Wallis with post-hoc comparisons. ns=not significant

FIG. 13 shows additional UMAPs and classifier performance for single NK cell OMI. (A) UMAP of NK cells with labels for both CD69+ and CD69− cells in the control and activated (IL-12+IL-15+IL-18) groups. (B) UMAP of NK cells color-coded by donor (D, E, F) and activation status (CD69+, CD69−). (C) Confusion matrix of the 9 OMI parameter random forest classifier trained to classify NK cells as CD69+ activated or CD69− control cells. (D) Confusion matrix of a logistic regression classifier trained on 9 OMI parameters to classify NK cells as CD69+ activated or CD69− control. (E) Confusion matrix of SVM classifier trained on 9 OMI parameters to classify NK cells as CD69+ activated or CD69− control. (F) ROC curves for the random forest, logistic regression, and SVM classifiers trained on 9 OMI parameters, with operating points indicated. In (A), n=1642 cells (554 activated CD69+ cells, 372 activated CD69-cells, 49 control CD69+ cells, 667 control CD69− cells). In (B)-(F), n=1221 cells (554 cells in the activated CD69+ condition, 667 cells in the control CD69− condition) with a 70/30 split for training and test sets.

FIG. 14 shows additional UMAPs and classifier performance for activation of lymphocytes (T cells, B cells and NK cells). (A) UMAP of single-cell OMI data from FIG. 9C containing all T, B, and NK cells color-coded by activation status. (B) Bar graph of % accuracy for a random forest classifier trained to distinguish CD69+ from CD69− cells across the combined dataset of all lymphocyte subtypes. (C) Pie chart displaying the weights of OMI variables included in the random forest classifier using all 9 OMI features in FIG. 9D. (D) ROC curves of random forest, logistic regression, and support vector matrix (SVM) classifiers using all 9 OMI variables to distinguish CD69+ from CD69− cells across all lymphocyte subtypes, with operating points indicated. (E) Confusion matrix for random forest classifier using all 9 OMI variables to classify cells as activated (CD69+) or quiescent (CD69−) in FIG. 9D. (F) Confusion matrix for logistic regression classifier. (G) Confusion matrix for SVM classifier. n=3127 cells (1747 CD69− cells, 1380 CD69+ cells) with a 50/50 split for training and test sets. T cell data taken from previously published dataset.

FIG. 15 shows additional UMAPs and classifier performance for lymphocyte subtype (T cells, B cells and NK cells). (A) UMAP of lymphocytes from FIG. 9C color-coded by lymphocyte subtype. (B) Variable weights of 9 OMI parameters used for one-vs.-one random forest classification by lymphocyte subtype in FIG. 9E. (C) Confusion matrix for 9 OMI parameter random forest classifier in FIG. 5E. n=3127 cells (1210 B cells, 1221 NK cells, 696 T cells) with a 50/50 split for training and test sets. T cell data taken from previously published dataset.

FIG. 16 shows UMAP and classifier performance for lymphocyte subtype classifier based on quiescent cells only (T cells, B cells and NK cells). (A) UMAP of lymphocytes from quiescent (CD69− control) NK, B, and T cells color-coded by lymphocyte subtype. (B) Bar graph displaying accuracy of random forest classifiers trained to separate lymphocytes based on lymphocyte subtype (one vs. one approach, quiescent cells only). (C) Feature weights of 9 OMI parameters used for one-vs.-one random forest classification by lymphocyte subtype in (B). (D) Confusion matrix for 9 OMI parameter random forest classifier in (B). n=1747 cells (749 B cells, 667 NK cells, 331 T cells) with a 50/50 split for training and test sets. T cell data taken from previously published dataset.

FIG. 17 shows additional UMAPs and classifier performance for both lymphocyte subtype (T cells, B cells and NK cells) and activation. (A) UMAP of lymphocytes from FIG. 9C color-coded by lymphocyte subtype, activation status, and donor. (B) Feature weights of 9 OMI parameters used for one-vs.-one random forest classification by lymphocyte subtype and activation status in FIG. 9F (C) Confusion matrix for 9 OMI parameter random forest classifier in FIG. 9F. n=3127 cells (749 CD69− control B cells, 461 CD69+ activated B cells, 667 CD69− control NK cells, 554 CD69+ activated NK cells, 331 CD69− control T cells, 365 CD69+ activated T cells) with a 50/50 split for training and test sets. T cell data taken from previously published dataset.

FIG. 18 shows phasor-based classification of NK cell and B cell activation and lymphocyte subtype. (A) NAD(P)H phasor plot of B cells from FIG. 5 (Red=activated CD69+ B cells, blue=quiescent CD69− B cells). Shaded areas show decision boundaries for logistic regression classification of B cell activation based on NAD(P)H phasor. (B) ROC curves and confusion matrix for random forest classification of B cell activation. The NAD(P)H and FAD phasors at both the laser repetition frequency (80 MHz) and its second harmonic (160 MHz) predicted B cell activation with a classification accuracy of 93.9%, n=1323 B cells (451 cells in the activated CD69+ condition, 872 cells in the control CD69− condition) with a 50/50 split for training and test sets. (C) NAD(P)H phasor plot of NK cells from FIG. 7 (Red=activated CD69+NK cells, blue=quiescent CD69− NK cells). Shaded areas show decision boundaries for logistic regression classification of NK cell activation based on NAD(P)H phasor. (D) ROC curves and confusion matrix for random forest classification of NK cell activation. The NAD(P)H and FAD phasors at both the laser repetition frequency (80 MHz) and its second harmonic (160 MHz) predicted NK cell activation with a classification accuracy of 89.2%, n=1742 cells (781 cells in the activated CD69+ condition, 961 cells in the control CD69− condition) with a 50/50 split for training and test sets. (E) Phasor plots of NAD(P)H (top) and FAD (bottom) of B cells and NK cells at both the laser repetition rate (80 MHZ) and its second harmonic (160 MHZ). (F) ROC curve and confusion matrix for logistic regression classification of B vs NK. Using both the NAD(P)H and FAD phasors at 80 MHz and 160 MHz, the logistic regression model could classify a cell as B or NK with a classification accuracy of 99.9%, n=3065 cells (1323 B cells, 1742 NK cells) with a 50/50 split for training and test sets. Due to separate processing pipelines, the total number of cells in phasor analysis differs from that in the fit analysis. Both analysis pipelines use the same raw data.

FIG. 19 shows phasor-based classification of NK cell, B cell, T cell lymphocyte subtype and activation. (A) NAD(P)H phasor plot of B cells, NK cells, and T cells. (B) Confusion matrix for random forest classification of lymphocyte subtype. The classifier was trained on NAD(P)H phasors (at the laser repetition frequency 80 MHz and its second harmonic 160 MHz) and achieves a classification accuracy of 96.2%. (C) Confusion matrix for random forest classification of lymphocyte subtype and activation trained on NAD(P)H phasors achieves a classification accuracy of 88.5%. The classifier predicted lymphocyte subtype and activation with a total n=3653 lymphocytes including n=1323 B cells (451 B cells in the activated CD69+ condition, 872 B cells in the control CD69− condition); n=1742 NK cells (781 cells in the activated CD69+ condition, 961 cells in the control CD69− condition); n=588 T cells (263 cells in the activated condition, 325 cells in the control condition); a 50/50 data split for training and test sets was used. Due to separate processing pipelines and exclusion criteria, the total number of cells in phasor analysis differs from that in the fit analysis. Both analysis pipelines use the same raw data. T cell data taken from previously published dataset.

FIG. 20 shows accuracies for classifiers given in figures. Accuracies are given out of a maximum of 1. For main FIG. 8D the top 1 feature is the redox ratio (RR).

### DETAILED DESCRIPTION

Before the present invention is described in further detail, it is to be understood that the invention is not limited to the particular embodiments described. It is also understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

Specific structures, devices and methods relating to modifying biological molecules are disclosed. It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

As used herein, the term “lymphocyte” refers to cells that are CD45+ and CD3+, CD19+, and/or CD56+.

As used herein, the term “B cell” refers to cells that are CD45+ and CD19+.

As used herein, the terms “natural killer” cell or “NK” cell refers to cells that are CD45+ and CD56+.

As used herein, the term “T cell” refers to cells that are CD45+ and CD3+.

As used herein, “cell size” refers to a measured geometric area of a cell of interest as determined by analyzing an acquired image of the cell of interest.

As used herein, the term “memory” includes a non-volatile medium, e.g., a magnetic media or hard disk, optical storage, or flash memory; a volatile medium, such as system memory, e.g., random access memory (RAM) such as DRAM, SRAM, EDO RAM, RAMBUS RAM, DR DRAM, etc.; or an installation medium, such as software media, e.g., a CD-ROM, or floppy disks, on which programs may be stored and/or data communications may be buffered. The term “memory” may also include other types of memory or combinations thereof.

As used herein, the term “FAD” refers to flavin adenine dinucleotide.

As used herein, the term “NAD(P)H” refers to reduced nicotinamide adenine dinucleotide and/or reduced nicotinamide dinucleotide phosphate.

As used herein, the term “processor” may include one or more processors and memories and/or one or more programmable hardware elements. As used herein, the term “processor” is intended to include any of types of processors, CPUs, GPUs, microcontrollers, digital signal processors, or other devices capable of executing software instructions.

As used herein, the term “redox ratio” or “optical redox ratio” refers to a ratio of NAD(P)H fluorescence intensity to FAD fluorescence intensity; a ratio of FAD fluorescence intensity to NAD(P)H fluorescence intensity; a ratio of NAD(P)H fluorescence intensity to any arithmetic combination including FAD fluorescence intensity; or a ratio of FAD fluorescence intensity to any arithmetic combination including NAD(P)H fluorescence intensity. In certain cases, the optical redox ratio or optical redox ratio refers to a ratio of NAD(P)H fluorescence intensity to the sum of NAD(P)H and FAD fluorescence intensity.

Autofluorescence endpoints include photon counts/intensity and fluorescence lifetimes. The fluorescence lifetime of cells can be a single value, the mean fluorescence lifetime, or compromised from the lifetime values of multiple subspecies with different lifetimes. In this case, multiple lifetimes and lifetime component amplitude values are extracted. Both NAD(P)H and FAD can exist in quenched (short lifetime) and unquenched (long lifetime) configurations; therefore, the fluorescence decays of NAD(P)H and FAD are fit to two components. Generally, NADH and FAD fluorescence lifetime decays are fit to a two component exponential decay, I(t)=α₁e^{-t/τ1}+α₂e^{-t/τ2}+C, where I(t) is the fluorescence intensity as a function of time, t, after the laser pulse, α₁ and α₂ are the fractional contributions of the short and long lifetime components, respectively (i.e., α₁+α₂=1), τ₁ and τ₂ are the short and long lifetime components, respectively, and C accounts for background light. However, the lifetime decay can be fit to more components (in theory any number of components, although practically up to ˜5-6) which would allow quantification of additional lifetimes and component amplitudes. By convention lifetimes τ and amplitudes α are numbered from short to long, but this notation could be reversed. A mean lifetime τₘ can be computed from the lifetime components, (τₘ=α₁τ1+α₂τ₂ . . . ). Fluorescence lifetimes and lifetime component amplitudes can also be approximated from frequency domain data collection and analysis and gated cameras/detectors. For gated detection, α₁ could be approximated by dividing the detected intensity at early time bins by later time bins. Alternatively, fluorescence anisotropy can be measured by polarization-sensitive detection of the autofluorescence, thus identifying free NAD(P)H as the short rotational diffusion time in the range of 100-700 ps.

FAD α₁ refers to the contribution of bound FAD and is the shortest lifetime that is not dominated (i.e., greater than 50%) by instrument response and/or scattering. FAD α₁ is the contribution associated with FAD lifetime values from 50-1500 ps, from 50-1000 ps, or from 50-600 ps. For clarity, a claim herein including features related to a “shortest” lifetime cannot be avoided by defining the lifetime values to include a sacrificial shortest lifetime that is dominated by instrument response and/or scattering.

FAD τ₁ refers to the bound FAD lifetime and is the shortest lifetime that is not dominated (i.e., greater than 50%) by instrument response and/or scattering. FAD τ₁ is the FAD lifetime values from 50-1500 ps, from 50-1000 ps, or from 50-600 ps. For clarity, a claim herein including features related to a “shortest” lifetime cannot be avoided by defining the lifetime values to include a sacrificial shortest lifetime that is dominated by instrument response and/or scattering.

FAD τ₂ refers to the free FAD lifetime and is the longest lifetime that is not dominated (i.e., greater than 50%) by instrument response and/or scattering. FAD τ₂ is the FAD lifetime values from 1000-4000 ps, from 1000-3000 ps, or from 1500-3000 ps. For clarity, a claim herein including features related to a “longest” lifetime cannot be avoided by defining the lifetime values to include a sacrificial shortest lifetime that is dominated by instrument response and/or scattering.

FAD τₘ=α₁·τ₁+(1−α₁)·τ₂

NAD(P)H α₁ refers to the contribution of free NAD(P)H and is the shortest lifetime that is not dominated (i.e., greater than 50%) by instrument response and/or scattering. NAD(P)H α₁ is the contribution associated with NAD(P)H lifetime values from 50-1500 ps, from 50-1000 ps, or from 50-600 ps. For clarity, a claim herein including features related to a “shortest” lifetime cannot be avoided by defining the lifetime values to include a sacrificial shortest lifetime that is dominated by instrument response and/or scattering.

NAD(P)H τ₁ refers to the free NAD(P)H lifetime and is the shortest lifetime that is not dominated (i.e., greater than 50%) by instrument response and/or scattering. NAD(P)H τ₁ is the NAD(P)H lifetime values from 200-1500 ns, from 200-1000 ns, or from 200-600 ns. For clarity, a claim herein including features related to a “shortest” lifetime cannot be avoided by defining the lifetime values to include a sacrificial shortest lifetime that is dominated by instrument response and/or scattering.

NAD(P)H τ₂ refers to the bound NAD(P)H lifetime and is the longest lifetime that is not dominated (i.e., greater than 50%) by instrument response and/or scattering. NAD(P)H τ₂ is the NAD(P)H lifetime values from 1000-4000 ns, from 1000-3000 ns, or from 1500-3000 ns. For clarity, a claim herein including features related to a “longest” lifetime cannot be avoided by defining the lifetime values to include a sacrificial shortest lifetime that is dominated by instrument response and/or scattering.

NAD(P)H τₘ=α₁·τ₁+(1−α₁)·τ₂

The various aspects may be described herein in terms of various functional components and processing steps. It should be appreciated that such components and steps may be realized by any number of hardware components configured to perform the specified functions.

### Methods

This disclosure provides a variety of methods. It should be appreciated that various methods are suitable for use with other methods. Similarly, it should be appreciated that various methods are suitable for use with the systems described elsewhere herein. When a feature of the present disclosure is described with respect to a given method, that feature is also expressly contemplated as being useful for the other methods and systems described herein, unless the context clearly dictates otherwise.

The methods described herein can provide predictions that relate to activation status and/or lymphocyte type of a given lymphocyte. Each of the described predictions can be performed in parallel with other predictions, so the descriptions are not intended to be mutually exclusive, unless the context clearly dictates otherwise. Given that the methods are non-destructive, all of the methods described herein can be performed on a given cell, unless expressly limited by the nature of the cell and method (e.g., if a given cell is a B cell and the method is specific to cells that are known to be NK cells, then the method cannot be performed).

In some cases, the methods described herein include predictions regarding identifying the activation status of a given lymphocyte. This prediction is a current activation prediction, which provides a computer-generated prediction for the current state of activation in a given lymphocyte of interest. For example, the current activation prediction may indicate that a given lymphocyte is activated or it may indicate that the cell is quiescent. The given lymphocyte may have an unknown lymphocyte type, so the methods may in some cases be unable to rely on parameters that depend on knowing whether the given cell is a B cell, a NK cell, or a T cell.

In some cases, the methods described herein includes predictions regarding identifying the specific lymphocyte identification of a given lymphocyte. The prediction is a current identification prediction, which provides a computer-generated prediction for the current lymphocyte identification of a given lymphocyte of interest. For example, the current identification prediction may indicate that a given lymphocyte is a T cell (or a NK cell or a B cell). In this prediction the given lymphocyte can have an unknown lymphocyte type, so the methods cannot rely on parameters that depend on knowing whether the given cell is a B cell, a NK cell, or a T cell.

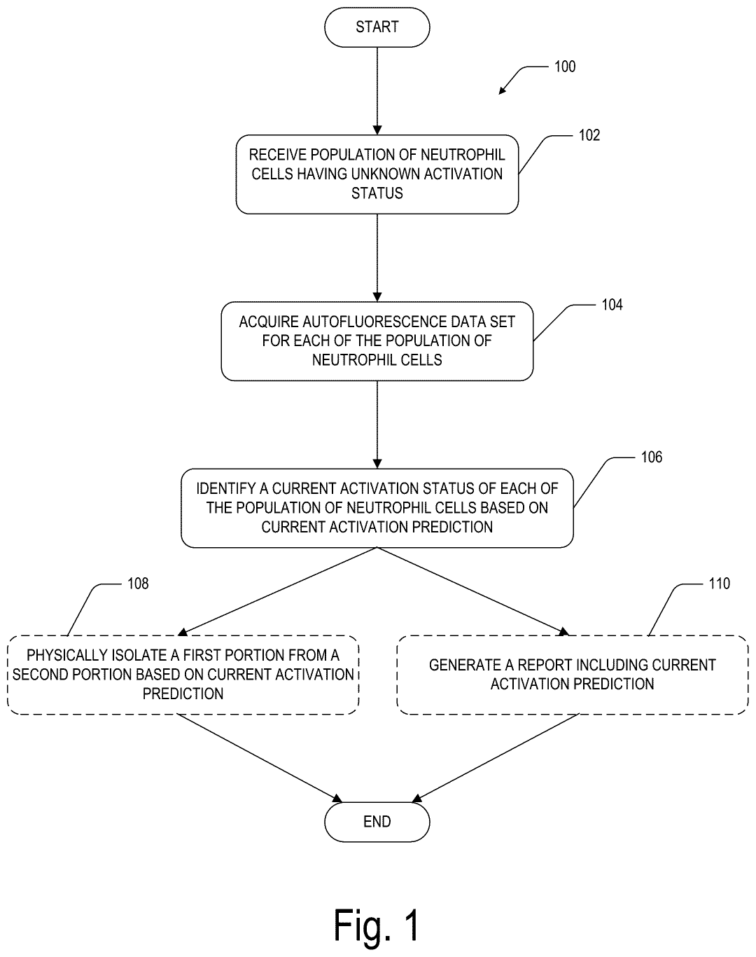

Referring to FIG. 1, the present disclosure provides a method 100 of characterizing lymphocyte activation and/or identification status. At optional process block 102, the method 100 optionally includes receiving a population of lymphocytes having unknown activation status and unknown lymphocyte type (i.e., unknown whether a given lymphocyte is a B cell, a NK cell, or a T cell). The population of lymphocytes can itself be contained within a broader population of cells that includes some cells that are not lymphocytes. At process block 104, the method 100 includes acquiring an autofluorescence data set for each lymphocyte of the population of lymphocytes. At process block 106, the method 100 includes identifying a current activation and/or identification status of each of the lymphocytes based on a current activation and/or identification prediction. The current activation and/or identification prediction is computed using at least a portion of the autofluorescence data set. The current activation and/or identification prediction is computed using at least one metabolic endpoint of the autofluorescence data set as an input. The at least one metabolic endpoint includes those described below. Following process block 106, the method 100 can proceed to process block 108 or 110, depending on the desired outcome. In some cases, the method 100 proceeds to process block 108 and process block 110, in either order. While process blocks 108 and 110 are both illustrated and described as optional, the method 100 includes either process block 108 or process block 110. At optional process block 108, the method 100 optionally includes physically isolating a first portion of the population of lymphocytes from a second portion of the population of lymphocytes based on a current activation prediction, wherein each lymphocyte of the population of lymphocytes is placed into the first portion when the current activation prediction exceeds a predetermined threshold and into the second portion when the current activation prediction is less than or equal to the predetermined threshold. At optional process block 110, the method 100 optionally includes generating a report including the current activation prediction. The report optionally includes identifying a proportion of the population of lymphocytes having a current activation and/or identification prediction that exceeds a predetermined threshold.

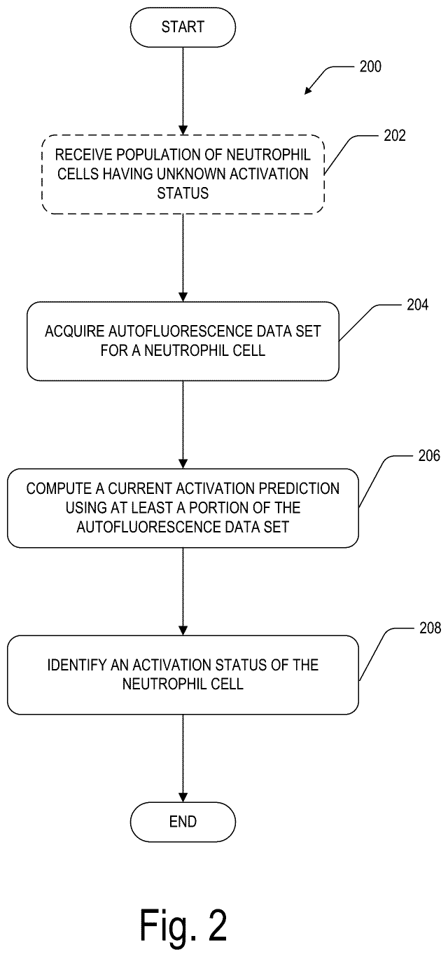

Referring to FIG. 2, the present disclosure provides a method 200 of characterizing lymphocyte activation status. At optional process block 202, the method 200 optionally includes receiving a population of lymphocytes having unknown activation status. At process block 204, the method 200 includes acquiring an autofluorescence data set from a lymphocyte of the population of lymphocytes. At process block 206, the method 200 includes computing a current activation prediction using at least a portion of the autofluorescence data set. The current activation prediction is computed using at least one metabolic endpoint. The at least one metabolic endpoint can include those outlined below. At process block 208, the method 200 includes identifying a current activation status of the lymphocyte based on the current activation prediction.

Method 100 and method 200 are related to one another and can be utilized together. For example, method 200 can be utilized within method 100. Aspects described with respect to method 100 can be utilized in method 200, unless the context clearly dictates otherwise, and vice versa.

The autofluorescence data set acquired at process block 104 or 204 can be acquired in a variety of ways, as would be understood by one having ordinary skill in the spectroscopic arts with knowledge of this disclosure and their own knowledge from the field. For example, the autofluorescence data can be acquired from fluorescence decay data. As another example, the autofluorescence data can be acquired by gating a detector (a camera, for instance) to acquire data at specific times throughout a decay in order to approximate the autofluorescence endpoints described herein. As yet another example, a frequency domain approach can be used to measure and analyze lifetime. Alternatively, fluorescence anisotropy can be measured by polarization-sensitive detection of the autofluorescence, thus identifying free NAD(P)H as the short rotational diffusion time in the range of 100-700 ps. The specific way in which autofluorescence data is acquired is not intended to be limiting to the scope of the present invention, so long as the lifetime information necessary to determine the autofluorescence endpoints necessary for the methods described herein can be suitably measured, estimated, or determined in any fashion. One example of a suitable autofluorescence data set acquisition is described below in the Examples section.

The physical isolation operation of optional process block 108 is in response to a current activation prediction determined from the acquired autofluorescence data set. If the current activation prediction exceeds a predetermined threshold for a given lymphocyte, then that lymphocyte is placed into the first portion. If the current activation prediction is less than or equal to the predetermined threshold for the given lymphocyte, then that lymphocyte is placed into the second portion. The result of this physical isolation is that the first portion of the population of lymphocytes is significantly enriched in lymphocytes having a given activation status (e.g., activated or quiescent), whereas the second portion of the population of lymphocytes is significantly depleted of lymphocytes having that given activation status.

In some cases, the physical isolation operation of optional process block 108 can include isolating cells into three, four, five, six, or more portions. In these cases, the different portions will be separated by a number of predetermined thresholds that is one less than the number of portions (i.e., three portions=two predetermined thresholds). The portion whose current activation prediction exceeds all of the predetermined thresholds (i.e., exceeds the highest threshold) contains the greatest concentration of lymphocytes with a given activation status. The portion whose current activation prediction fails to exceed any of the predetermined thresholds (i.e., fails to exceed the lowest threshold) contains the lowest concentration of lymphocytes with the given activation status. Using multiple predetermined thresholds can afford the preparation of portions of the population of lymphocytes that have extremely high or extremely low concentrations of lymphocytes with the given activation status. In some cases, the physical isolation operation of optional process block 108 (or a totally separate aspect of method 100, as would be appreciated by those having ordinary skill in the cell isolation arts) can include isolating other kinds of cells, such as red blood cells or the like, or various kinds of debris so they are not included in the portions including lymphocytes.

The current activation prediction is computed using at least one metabolic endpoint of the autofluorescence data set for each lymphocyte of the population of lymphocytes as an input. The at least one metabolic endpoint typically includes reduced nicotinamide adenine dinucleotide and/or reduced nicotinamide dinucleotide phosphate (NAD(P)H) shortest fluorescence amplitude component (α₁). The current activation prediction is computed using an equation that is generated by a machine learning process on data for a population of lymphocytes having a known activation status using the at least one metabolic endpoint and as variable(s).

The at least one metabolic endpoint can include one or more of the following: NAD(P)H fluorescence intensity; NAD(P)H shortest lifetime amplitude component or NAD(P)H α₁; NAD(P)H mean fluorescence lifetime or NAD(P)H τₘ; NAD(P)H shortest fluorescence lifetime or NAD(P)H τ₁; NAD(P)H second shortest fluorescence lifetime or NAD(P)H τ₂; an optical redox ratio (e.g., NAD(P)H/[NAD(P)H+FAD], see definition above); FAD fluorescence intensity; FAD mean fluorescence lifetime or FAD τₘ, the FAD shortest fluorescence amplitude component or FAD α₁, the FAD shortest fluorescence lifetime component or FAD τ₁, the FAD longest fluorescence lifetime component or FAD τ₂, or a combination thereof. The relative weightings of the at least one metabolic endpoints can vary based on the particular cell type and prediction and are discussed below with respect to more specific aspects.

In certain cases, the at least one metabolic endpoint can include, in no particular order, the NAD(P)H mean fluorescence lifetime or NAD(P)H τₘ; the FAD shortest fluorescence lifetime component (τ₁); FAD mean fluorescence lifetime (τₘ); NAD(P)H shortest lifetime amplitude component or NAD(P)H α₁; the FAD shortest lifetime amplitude component (α₁); an optical redox ratio (e.g., NAD(P)H/[NAD(P)H+FAD], see definition above); NAD(P)H shortest fluorescence lifetime or NAD(P)H τ₁; the FAD longest fluorescence lifetime component (τ₂), NAD(P)H second shortest fluorescence lifetime or NAD(P)H τ₂; or a combination thereof. The at least one metabolic endpoint can also optionally include one or more of the following: NAD(P)H fluorescence intensity or FAD fluorescence intensity.

The at least one morphological parameter can include solidity, eccentricity, an area of the lymphocyte, a perimeter of the lymphocyte, convex area which is the area of the convex hull (i.e., the smallest convex polygon that fits around the cell) that encloses a lymphocyte, major axis length or a combination thereof.

In some cases, the current activation prediction can be computed using cell size as an input. However, the inventors unexpectedly discovered that including cell size in computing the current activation prediction provided little improvement in prediction quality. As a result, while the present disclosure is intended to encompass embodiments that do measure cell size and include that cell size measurement in the various predictions that are made, the present disclosure expressly contemplates excluding cell size. The exclusion of cell size can be preferential in some cases, because it allows predictions to be made in circumstances where measuring cell size may not be practical.

In some cases, two, three, four, five, six, seven, eight, nine, ten, eleven, twelve, thirteen, fourteen, or more inputs are used.

In some cases, a surprisingly small number of metabolic endpoints and morphological parameters can be used while still achieving an adequate level of classification accuracy, as described below.

In some cases, the predictions described herein are computed using a phasor analysis, as described in International Patent Application Pub. No. 2021/232011, which is incorporated herein in its entirety by reference for all purposes. Briefly, a first phasor at a first frequency and a second phasor at a second, different frequency are computed from the time-resolved autofluorescence decay, and then the activation prediction can be computed using these phasors.

The method 100 or method 200 can sort lymphocytes into the categories of activated and quiescent based on the current activation status.

The method 100 or method 200 can sort lymphocytes into the categories of T cell, B cell, and NK cell based on the current identification status.

The method 100 or method 200 can provide surprising accuracy of classifying lymphocyte current activation or identification state. The accuracy can be at least 70%, at least 72.5%, at least 75%, at least 77.5%, at least 80%, at least 82.5%, at least 85%, at least 87.5%, at least 90%, at least 92.5%, at least 95%, at least 96%, at least 97%, at least 98%, or at least 99%. One non-limiting example of measuring the accuracy includes executing the method 100 or method 200 on a given cell with unknown current activation status and then using one of the traditional methods for determining activation status (which will typically be a destructive method) for a number of cells that is statistically significant.

The method 100 or method 200 can be performed without the use of a fluorescent label for binding the lymphocyte. The method 100 or method 200 can be performed without immobilizing the lymphocyte.

In certain cases, the method 100 or method 200 is performed on lymphocytes having unknown identity status.

In some cases, the method 100 or method 200 is used to perform a six-class classification, which can identify lymphocytes as activated T cells, quiescent T cells, activated B cells, quiescent B cells, activated NK cells, or quiescent NK cells. The six-class classification is computed using at least three six-class metabolic endpoints of the autofluorescence data set. The at least three six-class metabolic endpoints include reduced nicotinamide adenine dinucleotide and/or reduced nicotinamide dinucleotide phosphate (NAD(P)H) shortest fluorescence amplitude component (α₁), NAD(P)H shortest fluorescence lifetime component (τ₁), and NAD(P)H mean fluorescence lifetime (τₘ).

We used random forest classification to classify both cell type and activation simultaneously. A six-class classification was performed (one vs. one approach). Again, the classifier with all 9 metabolic endpoints had the highest accuracy (91.7%), but other classifiers also performed well. In one case, the top four parameters (NAD(P)H α₁, NAD(P)H τ₁, NAD(P)H τₘ, FAD τₘ) gave an accuracy of 88.7%. In another case, the top three parameters (NAD(P)H α₁, NAD(P)H τ₁, NAD(P)H τₘ) gave an accuracy of at least 86%. In another case, the NAD(P)H lifetime variables (τₘ, τ₁, τ₂, α₁) provided an accuracy of 87.2%.

A confusion matrix reveals that the majority of misclassification was between whether a cell was activated or quiescent, while cell type was usually identified correctly.

In some cases, the six-class classification is capable of being computed or is computed using only data secured with a single wavelength of light. In some cases, the six-class classification is capable of being computed or is computed using only NAD(P)H α₁, NAD(P)H τₘ, and NAD(P)H τ₁ as the input to provide an accuracy of at least 86%. In some cases, the six-class classification is capable of being computed or is computed using only NAD(P)H α₁, NAD(P)H τₘ, NAD(P)H τ₁, and NAD(P)H τ₂ as the input to provide an accuracy of at least 87%. In some cases, the six-class classification is capable of being computed or is computed using only NAD(P)H α₁, NAD(P)H τₘ, NAD(P)H τ₁, NAD(P)H τ₂, FAD τ₁, FAD τₘ, FAD α₁, FAD τ₂, and optical redox ratio as the input to provide an accuracy of at least 91.5%.

The six-class classification can in some cases be used purely for the data that is collected. In other words, the analyzed cells may be superfluous and can be discarded after their use. However, this will not always be the case, as one of the most exciting applications of this technology includes administering cells that have been identified by the predictions disclosed herein. This administering is discussed in greater detail below.

In some cases, the method 100 or method 200 is used to distinguish between lymphocytes based on identity, which can identify lymphocytes as T cells, B cells, or NK cells. The lymphocyte identification prediction is computed using at least two lymphocyte identification metabolic endpoints as an input. The at least two lymphocyte identification metabolic endpoints include NAD(P)H α₁.

In one case, we investigated whether machine learning could be used to classify cell type with the combined lymphocyte data. Three-class random forest classification of lymphocyte cell type was performed (one vs. one approach) using different combinations of metabolic endpoints. In one example, classification with all nine metabolic endpoints provided an accuracy of 99.1%. The feature weights are FAD τₘ (28.5%), FAD τ₁ (26.3%), NAD(P)H τₘ (16.5%), FAD α₁ (10.3%), NAD(P)H τ₁ (8.3%), optical redox ratio (4.7%), NAD(P)H α₁ (3.7%), FAD τ₂ (0.9%), and NAD(P)H τ₂ (0.8%). In another example, the top four parameters resulted in a cell type identification accuracy of 97.1%. In another example, NAD(P)H lifetime variables, NAD(P)H τₘ, α₁, τ₁, and τ₂ gave an accuracy of 95.5%.

In some cases, the lymphocyte identification prediction is capable of being computed or is computed using only a single wavelength of excitation light. In some cases, the lymphocyte identification prediction is capable of being computed or is computed using only NAD(P)H α₁, NAD(P)H τₘ, NAD(P)H τ₁, and NAD(P)H τ₂ as the input to provide an accuracy of at least 95.5%. In some cases, the lymphocyte identification prediction is capable of being computed or is computed using only FAD τₘ, FAD τ₁, NAD(P)H τₘ, and FAD α₁ as the input to provide an accuracy of at least 97%. In some cases, the lymphocyte identification prediction is capable of being computed or is computed using only NAD(P)H α₁, NAD(P)H τₘ, NAD(P)H τ₁, NAD(P)H τ₂, FAD τ₁, FAD τₘ, FAD α₁, FAD τ₂, and optical redox ratio as the input to provide an accuracy of at least 99%.

In some cases, the method 100 or method 200 is used to distinguish between lymphocytes based on activity status, which can identify activated lymphocytes and quiescent lymphocytes, without requiring knowledge of which specific type of lymphocyte is being analyzed. The lymphocyte activation prediction is computed using at least two lymphocyte activation metabolic endpoints of the autofluorescent data set as an input. The at least two lymphocyte activation metabolic endpoints include NAD(P)H α₁ and one of an optical redox ratio, NAD(P)H τₘ, or NAD(P)H τ₁.

In one case, we investigated whether machine learning could be used to classify activation with the combined lymphocyte data. First, random forest classification was used to identify whether cells were activated (CD69+) or quiescent (CD69−). Using all 9 metabolic endpoints, an accuracy of 92.7% was achieved. The feature weights were NAD(P)H α₁ (26.0%), NAD(P)H τ₁ (17.8%), optical redox ratio (16.9%), NAD(P)H τm (9.73%), NAD(P)H τ₂ (7.6%), FAD α₁ (7.3%), FAD τ1 (5.9%), FAD τ2 (4.5%), and FAD τm (4.3%). In another example, classification of activation status based on NAD(P)H variables also had high performance (accuracy=90.5%). In yet another example, logistic regression and support vector machine classification of activation status provided accuracies of 81.4% and 82.2% respectively.

In some cases, the lymphocyte activation prediction is capable of being computed or is computed using a single wavelength of excitation light. In some cases, the lymphocyte activation prediction is capable of being computed or is computed using only NAD(P)H α₁ and NAD(P)H τ1 as the input to provide an accuracy of at least 86%. In some cases, the lymphocyte activation prediction is capable of being computed or is computes using only NAD(P)H α₁, NAD(P)H τ1, and an optical redox ratio as the input to provide an accuracy of at least 89%. In some cases, the lymphocyte activation prediction is capable of being computed or is computes using only NAD(P)H α₁, NAD(P)H τ1, an optical redox ratio, and NAD(P)H τₘ as the input to provide an accuracy of at least 90%. In some cases, the lymphocyte activation prediction is capable of being computed or is computes using only NAD(P)H α₁, NAD(P)H τₘ, NAD(P)H τ₁, and NAD(P)H τ₂ as the input to provide an accuracy of at least 90%. In some cases, the lymphocyte activation prediction is capable of being computed or is computes using only NAD(P)H α₁, NAD(P)H τₘ, NAD(P)H τ₁, NAD(P)H τ₂, FAD α₁, FAD τₘ, FAD τ₁, FAD τ₂, and optical redox ratio as the input to provide an accuracy of at least 92%.

In certain cases, the method 100 or method 200 is performed on B cells and can distinguish between activated and quiescent B cells. The B cell activation prediction is computed using at least two B cell activation metabolic endpoints of the autofluorescence data set as an input. The at least two B cell activation metabolic endpoints includes NAD(P)H α₁ and NAD(P)H τₘ.

In some cases, the B cell activation prediction is capable of being computed or is computed using only the NAD(P)H α₁ and the NAD(P)H τₘ as the input to provide an accuracy of at least 86%. In some cases, the B cell activation prediction is capable of being computed or is computed using only the NAD(P)H α₁, the NAD(P)H τₘ, and the FAD τ₂ as the input to provide an accuracy of at least 89%. In some cases, the B cell activation prediction is capable of being computed or is computed using only the NAD(P)H α₁, the NAD(P)H τₘ, the FAD τ₂, and the NAD(P)H τ₁ as the input to provide an accuracy of at least 90%. In some cases, the B cell activation prediction is capable of being computed or is computed using only the NAD(P)H α₁, the NAD(P)H τₘ, the NAD(P)H τ₁, the NAD(P)H τ₂, the FAD α₁, the FAD τₘ, the FAD τ₁, the FAD τ₂, and optical redox ratio as the input to provide an accuracy of at least 92%.

In certain cases, the method 100 or method 200 is performed on NK cells. The NK cell activation prediction is computed using at least two NK cell activation metabolic endpoints of the autofluorescence data set as an input. The at least two NK activation metabolic endpoints include either: NAD(P)H α₁ and an optical redox ratio; or NAD(P)H α₁, NAD(P)H τₘ, and NAD(P)H second shortest lifetime (τ₂), and NAD(P)H τ₁.

In some cases, the NK cell activation prediction is capable of being computed or is computed using only the NAD(P)H α₁ and an optical redox ratio as the input to provide an accuracy of at least 81%. In some cases, the NK cell activation prediction is capable of being computed or is computed using only the NAD(P)H α₁, an optical redox ratio, and the NAD(P)H τ₂ as the input to provide an accuracy of at least 89%. In some cases, the NK cell activation prediction is capable of being computed or is computed using only the NAD(P)H α₁, an optical redox ratio, the NAD(P)H τ₂, and the NAD(P)H τ₁ as the input to provide an accuracy of at least 92%. In some cases, the NK cell activation prediction is capable of being computed or is computed using only the NAD(P)H α₁, the NAD(P)H τₘ, the NAD(P)H τ₁, and the NAD(P)H τ₂ as the input to provide an accuracy of at least 91.5%. In some cases, the NK cell activation prediction is capable of being computed or is computed using only the NAD(P)H α₁, the NAD(P)H τₘ, the NAD(P)H τ₁, the NAD(P)H τ₂, the FAD α₁, the FAD τₘ, the FAD τ₁, the FAD τ₂, and an optical redox ratio as the input to provide an accuracy of at least 92%.

In yet another case, we classified B cell and NK cell activation and cell type based on phasors. Logistic regression classifiers were trained on NADH and FAD phasors at the laser repetition rate (80 MHz) and its second harmonic (160 MHZ). Classification based on the NADH phasor at the laser repetition frequency (80 MHz) predicted B cell activation with 93.4% accuracy and NK cell activation with 89.2% accuracy. Classification using both NADH and FAD 80 MHz and 160 MHz phasors could classify cells as either B or NK cells with 99.9% accuracy.

In some cases, two, three, four, five, six, seven, eight, nine, ten, eleven, twelve, thirteen, fourteen, or more inputs are used to classify lymphocyte cell type or activation status.

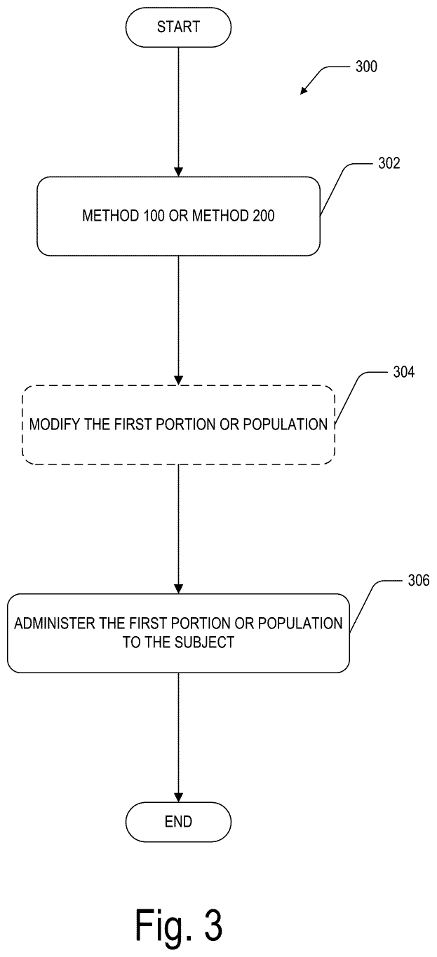

Referring to FIG. 3, the present disclosure provides a method 300 of administering activated lymphocytes to a subject in need thereof. At process block 302, the method 300 includes the method 100 or method 200 described above, which results in a first portion of the population of lymphocytes enriched for current activation and/or identification state (when optional process block 108 is utilized) or results in a report identifying the proportion of lymphocytes that have a given current activation and/or identification state (when optional process block 110 is utilized). At optional process block 304, the method 300 optionally includes modifying the first portion of the population of lymphocytes or the population of lymphocytes. The modifying can include gene editing. At process block 306, the method 300 includes administering the first portion of the population of lymphocytes, if the cells have been sorted, or the population of lymphocytes, if the cells have not been sorted, to the subject.

The lymphocytes can be harvested from a donor or, alternatively, from the subject to which they are administered prior to sorting. The lymphocytes can be either directly introduced to the subject or can undergo additional processing prior to introduction to the subject. In a non-limiting example, lymphocytes can be modified by viral means or by CRISPR, or stimulated by factors that make them potent against disease when administered back to the patient. In another non-limiting example, tumor infiltrating lymphocyte therapy can use cytokine stimulation or metabolic manipulation to enhance lymphocyte activities. A skilled artisan will recognize that a variety of modifications can be made to the cells, either genetic modification or stimulation with factors, which make the cells potent against disease when administered back to patient. The degree or extent of modification is not intended to be limiting to this disclosure, as the disclosure is principally related to identification of the cells earlier in the workflow.

The methods described herein provided surprising results to the inventors. Prior measurements of autofluorescence lifetimes in lymphocytes (Yakimov et al., Biomedical Optics Express 10(8) 1 Aug. 2019) show no separation between lymphocytes (T cells, B cells, NK cells) indicating it should not be possible to classify lymphocytes with autofluorescence lifetimes. Nothing in the literature reports B cell or NK cell activation measured by autofluorescence lifetime imaging. However, T cells, B cells, and NK cells are activated through different mechanisms and have very different functions, so it is surprising, based on prior T cell work by the inventors, that the NAD(P)H lifetimes would be sensitive to activation in B cells and NK cells.

A surprising result is that T cells, NK cells, and B cells can be classified from each other, both in their quiescent and activated states. All prior work indicates this is not likely.

### Systems

This disclosure also provides systems. The systems can be suitable for use with the methods described herein. When a feature of the present disclosure is described with respect to a given system, that feature is also expressly contemplated as being combinable with the other systems and methods described herein, unless the context clearly dictates otherwise.

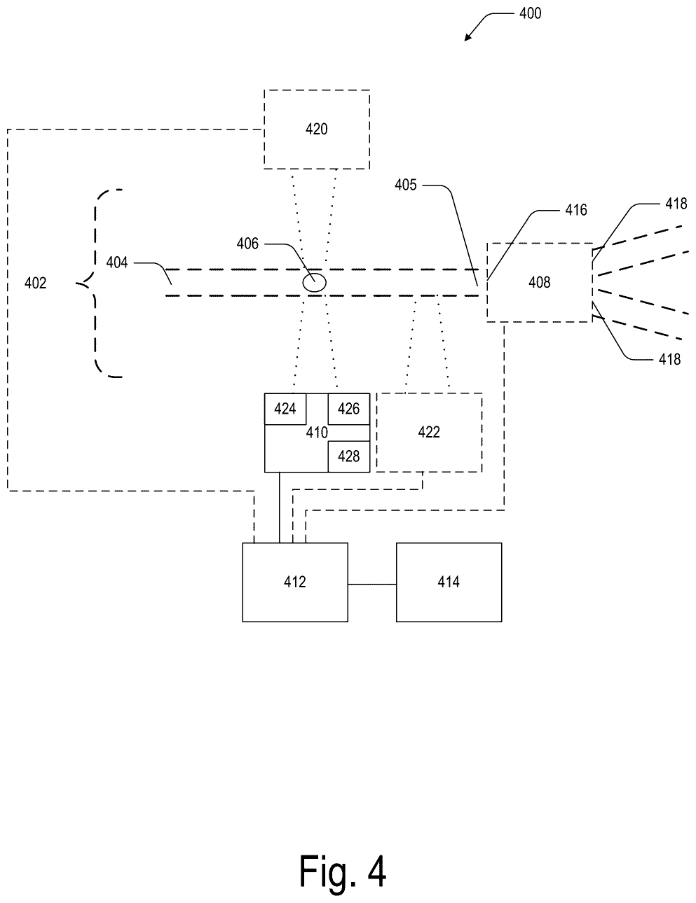

Referring to FIG. 4, the present disclosure provides a lymphocyte activation status sensing device 400. The device 400 includes an observation zone 406. The observation zone 406 is adapted to receive a cell analysis pathway 402, a cell culture (not illustrated), or other device or system capable of presenting lymphocytes for optical interrogation. The device 400 includes a processor 412 and a non-transitory computer-readable medium 414, such as a memory. In some configurations, the processor 412 can be or otherwise include a field-programmable gate array (FPGA). In configurations where the processor 412 is an FPGA, an additional processor (not shown) may be included to capture images.

The device 400 optionally includes a cell analysis pathway 402. The cell analysis pathway 402 includes an inlet 404, the observation zone 406, and an outlet 405. The device 400 optionally includes a cell sorter 408. The observation zone 406 is coupled to the inlet 404 downstream of the inlet 404 and is coupled to the outlet 405 upstream of the outlet 405. The device 400 also includes a single-cell autofluorescence spectrometer 410. The device 400 can further include an optional cell picker (not illustrated).

The inlet 404 can be any nanofluidic, microfluidic, or other cell sorting inlet. A person having ordinary skill in the art of fluidics has knowledge of suitable inlets 404 and the present disclosure is not intended to be bound by one specific implementation of an inlet 404.

The outlet can be any nanofluidic, microfluidic, or other cell sorting outlet. A person having ordinary skill in the art of fluidics has knowledge of suitable outlets 405 and the present disclosure is not intended to be bound by one specific implementation of an outlet 405.

The observation zone 406 is configured to present lymphocytes for individual autofluorescence decay interrogation. A person having ordinary skill in the art has knowledge of suitable observation zones 406 and the present disclosure is not intended to be bound by one specific implementation of an observation zone 406.

The optional cell sorter 408 has a sorter inlet 416 and at least two sorter outlets 418. The cell sorter is coupled to the observation zone 406 via the sorter inlet 416 downstream of the observation zone 406. The cell sorter 408 is configured to selectively direct a cell from the sorter inlet 416 to one of the at least two sorter outlets 418 based on a sort signal.

The inlet 404, observation zone 406, outlet 405, and optional cell sorter 408 can be components known to those having ordinary skill in the art to be useful in high-throughput cell screening devices or flow sorters, including commercial flow sorters. The cell analysis pathway 402 can further optionally include a flow regulator, as would be understood by those having ordinary skill in the art. The flow regulator can be configured to provide flow of cells through the observation zone at a rate that allows the autofluorescence spectrometer 410 to acquire the autofluorescence data set. A useful review of the sorts of fluidics that can be used in combination with the present disclosure is Shields et al., “Microfluidic cell sorting: a review of the advances in the separation of cells from debulking to rare cell isolation,” Lab Chip, 2015 Mar. 7; 15(5): 1230-49, which is incorporated herein by reference in its entirety.

The optional cell picker can serve a similar function as the optional cell sorter 408, namely, isolating cells based on a sort signal. The cell picker can be automated. One example of a suitable cell picker includes an ALS CellCelector™, available commercially from ALS Automated Lab Solutions GmbH, Jena, Germany.

The autofluorescence spectrometer 410 includes a light source 424, a photon-counting detector 426, and photon-counting electronics 428.

The autofluorescence spectrometer 410 can be any spectrometer suitable for acquiring autofluorescence data sets as understood by those having ordinary skill in the optical arts.

Suitable light sources 424 include, but are not limited to, lasers, LEDs, lamps, filtered light, fiber lasers, and the like. The light source 424 can be pulsed, which includes sources that are naturally pulsed and continuous sources that are chopped or otherwise optically modulated with an external component.

The light source 424 can provide pulses of light having a full-width at half maximum (FWHM) pulse width that is of a duration that is adequate to achieve the spectroscopic goals described herein, as would be appreciated by one having ordinary skill in the spectroscopic arts. In some cases, the FWHM pulse width is at least 1 fs, at least 5 fs, at least 10 fs, at least 25 fs, at least 50 fs, at least 100 fs, at least 200 fs, at least 350 fs, at least 500 fs, at least 750 fs, at least 1 ps, at least 3 ps, at least 5 ps, at least 10 ps, at least 20 ps, at least 50 ps, or at least 100 ps. In some cases, the FWHM pulse width is at most 10 ns, at most 1 ns, at most 900 ps, at most 750 ps, at most 600 ps, at most 500 ps, at most 400 ps, at most 250 ps, at most 175 ps, at most 100 ps, at most 75 ps, at most 60 ps, at most 50 ps, at most 35 ps, at most 25 ps, at most 20 ps, at most 15 ps, at most 10 ps, or at most 1 ps.

The light source 424 can emit wavelengths that are tuned to the absorption of NAD(P)H and/or FAD. In some cases, the wavelength is at least 340 nm, at least 345 nm, at least 350 nm, at least 355 nm, at least 360 nm, at least 365 nm, or at least 370 nm. In some cases, the wavelength is at most 415 nm, at most 410 nm, at most 405 nm, at most 400 nm, at most 395 nm, at most 390 nm, at most 385 nm, or at most 380 nm. In some cases, the wavelength is between 360 nm and 415 nm, between 350 nm and 410 nm, or between 370 nm and 380 nm. In some cases, the wavelength is 375 nm. In some cases, the wavelength is 2 times or 3 times these wavelength values (i.e., the frequency is ½ or ⅓). It should be appreciated that pulsed light sources inherently have some degree of bandwidth, so they are never exactly monochromatic. Thus, references herein to “wavelength” refer to either a wavelength at the peak intensity or a weighted average wavelength. In some cases, the pulsed light source 424 is a UV pulsed diode laser. In some cases, the pulsed light source has a wavelength that is double the peak absorption wavelength of NAD(P)H and/or FAD, with an ultrashort pulse duration, such that fluorescence excitation is achieved through two-photon excitation events, as understood by those having ordinary skill in the optical arts.

The photon-counting detector 426 can be any detector suitably capable of detecting single photons and delivering an analog or digital output representative of the detected photons. Examples of photon-counting detectors 426 include, but are not limited to, a photomultiplier tube, a photodiode, an avalanche photodiode, a single-photon avalanche diode (SPAD), a charge-coupled device, combinations thereof, and the like.

The photon-counting electronic 428 can include electronics understood by those having ordinary skill in the art to be suitable for use with single-photon detectors 426 to produce the data sets described herein. Examples of suitable photon-counting electronics 428 include, but are not limited to, a field-programmable gate array (FPGA), a dedicated digital signal processor (DSP) with a digitizer and a time-to-digital converter, a time-correlated single photon counting (TCSPC) electronic board with time-to-amplitude and analog-to-digital converter electronics (as implemented by Becker & Hickl, Berlin, Germany), combinations thereof, and the like.

The autofluorescence spectrometer 410 can be directly (i.e., the processor 412 communicates directly with the spectrometer 410 and receives the signals) or indirectly (i.e., the processor 412 communicates with a sub-controller that is specific to the spectrometer 410 and the signals from the spectrometer 410 can be modified or unmodified before sending to the processor 412) controlled by the processor 412. Autofluorescence data sets can be acquired by known spectroscopic methods. Fluorescence lifetime images can also be acquired by known imaging methods and those acquired images can be used by the systems and methods described herein, as would be understood by those having ordinary skill in the spectroscopic arts. The device 400 can include various optical filters tuned to isolate autofluorescence signals of interest. The optical filters can be tuned to the autofluorescence wavelengths of NAD(P)H and/or FAD.

The autofluorescence spectrometer 410 can be configured to acquire the autofluorescence dataset from the detector's 426 electrical output at a repetition rate understood by those having ordinary skill in the spectroscopic arts to be suitable for providing adequate sampling to observe the dynamics disclosed herein. In some cases, the repetition rate can be at least 1 kHz, at least 5 kHz, at least 10 kHz, at least 30 kHz, at least 50 kHz, at least 100 kHz, at least 500 kHz, at least 750 kHz, at least 1 MHZ, at least 4 MHZ, at least 7 MHz, at least 10 MHZ, at least 15 MHZ, at least 20 MHZ, at least 50 MHz, at least 100 MHz, at least 500 MHZ, or at least 1 GHz. In some cases, the repetition rate can be at most 1 THz, at most 800 GHz, at most 500 GHz, at most 250 GHz, at most 150 GHz, at most 100 GHz, at most 70 GHz, at most 50 GHz, at most 25 GHZ, at most 15 GHZ, at most 10 GHz, at most 6 GHZ, at most 2 GHZ, at most 1 GHz, at most 750 MHZ, at most 500 MHZ, at most 400 MHZ, at most 250 MHz, at most 175 MHz, or at most 100 MHz. While there can be downside associated with oversampling, in principle the present disclosure can function with as high of a sampling rate as can be achieved with existing technology. The repetition rates identified herein are based on the state of the art at the time the present disclosure was prepared and filed and are not intended to be limiting in the event that future developments facilitate a greater repetition rate.

The pulsed light source 424 can be configured to operate at pulse repetition rates that are adapted to acquire the needed fluorescence lifetime information. The maximum pulse repetition rate is limited by the fluorescence lifetime of the fluorophore of interest. The fluorescence decay must have fully died down by the time the next pulse of light is introduced to the sample in order to avoid ambiguity about the sources of data sets (i.e., was this particular fluorescent photon initiated by the most recent excitation pulse of light or the one preceding it?). The pulsed light source 424 can have a pulse repetition rate of up to 100 MHz, up to 80 MHz, up to 60 MHz, or up to 40 MHz. The lower limit of the pulse repetition rate is more practical in a sense of reducing the overall sampling time, but theoretically the data can be taken very slowly if there is some reason to do so.

The device 400 can optionally include an optical microscope 420 for acquiring visual images of cells that are located in the observation zone 406 or elsewhere along the cell analysis pathway 402.

The device 400 can optionally include a cell size measurement tool 422. The cell size measurement tool 422 can be any device capable of measuring the size of cells, including but not limited to, an optical microscope, such as optical microscope 420. In some cases, the optical microscope and the cell size measurement tool 422 are the same subsystem.

In some cases, the autofluorescence spectrometer 410 and the optical microscope 420 can be integrated into a single optical subsystem. In some cases, the autofluorescence spectrometer 410 and the cell size measurement tool 422 can be integrated into a single optical subsystem. While some aspects of the methods described herein can operate by not utilizing the cell size as an input to the convolutional neural network, it may be useful to measure the cell size for other purposes.

The processor 412 is in electronic communication with the spectrometer 410. The processor 412 is also in electronic communication with, when present, the optional cell sorter 408, the optional optical microscope 420, and the optional cell size measurement tool 422.

The non-transitory computer-readable medium 414 has stored thereon instructions that, when executed by the processor, cause the processor to execute at least a portion of the methods described herein. Equations for which the first and second phasor coordinates are inputs can also be stored on the non-transitory computer-readable medium 414. The non-transitory computer-readable medium 414 can be local to the device 400 or can be remote from the device, so long as it is accessible by the processor 412.

The device 400 can be substantially free of fluorescent labels (i.e., the cell analysis pathway 402 does not include a region for mixing the cell(s) with a fluorescent label). The device 400 can be substantially free of immobilizing agents for binding and immobilizing lymphocytes.

The present invention has been described in terms of one or more preferred embodiments, and it should be appreciated that many equivalents, alternatives, variations, and modifications, aside from those expressly stated, are possible and within the scope of the invention.

The invention will be more fully understood upon consideration of the following non-limiting examples.

### Example 1

### Abstract

New non-destructive tools are needed to reliably assess lymphocyte function for immune profiling and adoptive cell therapy. Optical metabolic imaging (OMI) is a label-free method that measures the autofluorescence intensity and lifetime of metabolic cofactors NAD(P)H and FAD to quantify metabolism at a single-cell level. Here, we investigate whether OMI can resolve metabolic changes between quiescent and activated primary human peripheral B cells and NK cells. We found that quiescent B and NK cells were more oxidized compared to activated cells. Additionally, the NAD(P)H mean fluorescence lifetime decreased and the fraction of free NAD(P)H increased in the activated B and NK cells compared to quiescent cells. Machine learning classified B cells and NK cells according to activation state (CD69+) based on OMI parameters with up to 94.2% and 92.6% accuracy, respectively. We added our previously published OMI data from activated and quiescent T cells and found that the NAD(P)H fluorescence lifetime increased in NK cells compared to T cells, and further increased in B cells compared to NK cells. Random forest models based on OMI classified lymphocytes according to subtype (B, NK, T cell) with 99.1% accuracy, and according to activation state (quiescent or activated) and subtype (B, NK, T cell) with 92.7% accuracy. Our results demonstrate that autofluorescence lifetime imaging can accurately assess lymphocyte activation and subtype in a label-free, non-destructive manner.

### Teaser

Label-free optical imaging can assess the metabolic state of lymphocytes on a single-cell level in a touch-free system.

### Introduction

Lymphocytes consist of natural killer (NK) cells, B cells, and T cells, and constitute approximately 20-40% of circulating white blood cells. T cells have diverse cytotoxic and immune-modulating activities after activation, and therapies that modulate T cell function are in development or clinical use for a range of diseases, including cancer, HIV, autoimmune disease, and transplant rejection. NK cells are cytotoxic and surveil the body for unhealthy cells. However, NK cells are not antigen-specific, and instead rely on a balance of activating and inhibitory signals to initiate cytotoxicity. Their cytotoxic function has led to particular interestin the role of NK cells in tumor cell clearance and in adoptive cell therapy for cancer. B cells, like T cells, are a part of the adaptive immune system, and their primary role is the production of antibodies. B cells are also antigen-presenting cells that can present peptides to T cells to promote their effector functions. Subtypes of B cells also secrete cytokines that can either attenuate or suppress the function of surrounding immune cells. The multiple functions of B cells provide several avenues for leveraging B cells as a platform for cell-based therapies, including antigen-presenting B cells as a cancer immunotherapy and protein production for rare genetic diseases. Immune profiling measures activation of NK, B, and T cells to astimulus (such as an antigen from a virus or bacterium), which can be used to identify individual response and potentially predict outcome. Therefore, the presence and activation of lymphocytes is important for monitoring immune health and response to therapy across a range of diseases.

New label-free and non-destructive tools are needed to assess lymphocyte activation and subtype in single cells. Single cell measurements capture lymphocyte heterogeneity within a patient, which significantly impacts prognosis. Non-destructive tools enable subsequent analysis and long-term study of cells, while label-free tools enable subsequent expansion and use of cells in patients, for example in adoptive cell therapy. Current methods to assess lymphocytes include flow cytometry, cytokine release, single-cell RNA sequencing, and cytometry by time of flight (CyTOF). Flow cytometry provides single-cell resolution, but requires labelling with fluorescent antibodies that can be time consuming, may be disruptive to cells, and complicates further use of cells. Bulk measurements of cytokine release are also popular but do not provide single-cell measurements, and ELISPOT, which provides single-cell cytokine release information also requires cell labeling. Additionally, cytokine based techniques cannot provide information about subsets of immune cells that do not secrete cytokines. Finally, single-cell RNA sequencing and CyTOF provide extensive single-cell information, but destroy the sample.

Optical metabolic imaging (OMI) is an attractive label-free tool to assess the metabolic state of single cells. OMI measures the autofluorescence intensity and lifetime of metaboliccofactors reduced nicotinamide adenine dinucleotide (phosphate) [NAD(P)H] and flavin adenine dinucleotide (FAD). The fluorescence of NADPH and NADH overlap, and are jointly referred to as NAD(P)H. Since only the reduced form of NADPH and NADH and the oxidized form of FAD are fluorescent, the fluorescence intensity ratio of NAD(P)H to FAD is defined as the “optical redox ratio”, which provides information about the overall redox state of the cell. NAD(P)H and FAD each have two distinct fluorescence lifetimes due to their free and protein-bound states, so fluorescence lifetime imaging (FLIM) provides insight into changes in free and protein-bound pools for each co-enzyme, along with changes in lifetimes due to environmental factors and preferred binding partners. OMI relies on endogenous fluorophores already present in cells, so it is minimally invasive and can provide nondestructive monitoring of cellular metabolism. Cell segmentation algorithms developed with OMI enable single-cell resolution, which provides insight into metabolic heterogeneity within the population.

OMI is a promising technique to evaluate lymphocyte activation and subtype because known metabolic shifts occur with activation and between NK, B, and T cells. Unstimulated NK, B, and T cells have low metabolic demands and largely rely on low levels of glycolysis and oxidative phosphorylation to generate ATP. Once activated, extra energy is needed to fuel the effector functions of lymphocytes. In order to fuel rapid proliferation and produce cytokines and other molecules, activated lymphocytes increase use of glucose through aerobic glycolysis and oxidative phosphorylation. Overnight stimulation with activating cytokines (including IL-2, IL-12, and IL-15) increases rates of glycolysis and oxidative phosphorylation in NK cells. Similar increases in glycolytic metabolism and oxidative phosphorylation occur with activation in B and T cells. While these three cell types share a close lineage, the metabolism of NK, B, and T cells are unique. In a study of splenic mouse T and B cells, resting T cells were found to have higher glucose uptake and lactate generation compared to resting B cells, with B cells showing higher mitochondrial mass than T cells. In T effector cells, fatty acid synthesis is necessary for differentiation and proliferation, but inhibition of this pathway in NK cells does not substantially impact their proliferation.

Previous work showed that OMI can classify primary human CD3⁺ and CD3⁺CD8⁺ T cells based on activation status. OMI has also been used to classify subsets of macrophages in monoculture, tumor coculture, and in vivo in zebrafish, and to distinguish between categories of blood cells (i.e. erythrocytes, monocytes, granulocytes, lymphocytes). Prior work also showed that NADH autofluorescence intensity increases in activated B cells compared to unstimulated B cells. These results demonstrate that OMI is promising for lymphocyte profiling, but to our knowledge, no prior study has built classifiers based on OMI for NK cell activation, B cell activation, or lymphocyte subtype. Given the relevance of NK cells, B cells, and T cells for cell therapy, immunotherapy, infectious disease, and immune profiling, this study investigates whether OMI can classify activation in NK cells and B cells, classify lymphocyte subtype (NK, B, T cells), and provide a six-group classifier for activation and lymphocyte subtype. These studies indicate that machine learning classifiers and label-free non-invasive OMI provide high accuracy for single cell classification of activation and lymphocyte subtype from primary human peripheral blood samples.

### Results

### OMI Resolves Metabolic Differences Between Quiescent and Activated Human B Cells

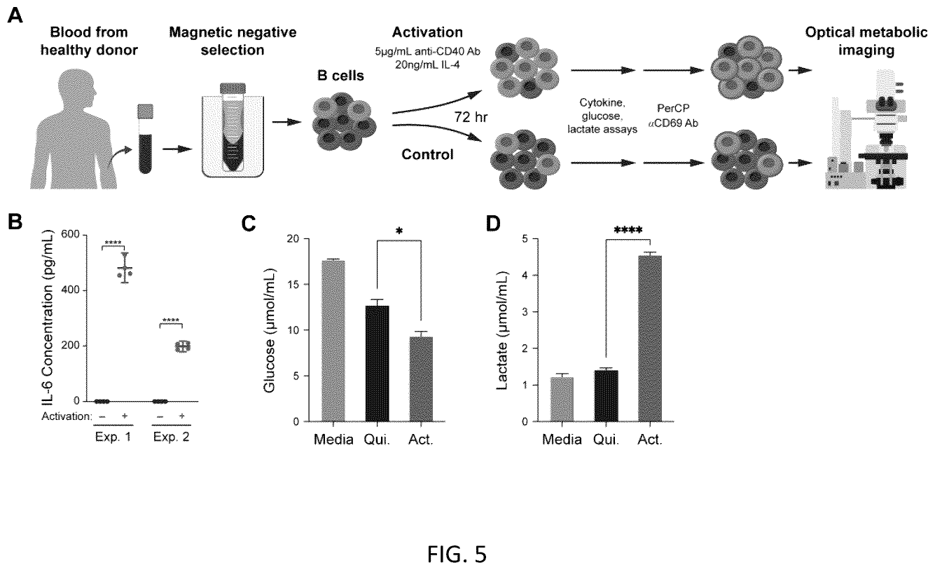

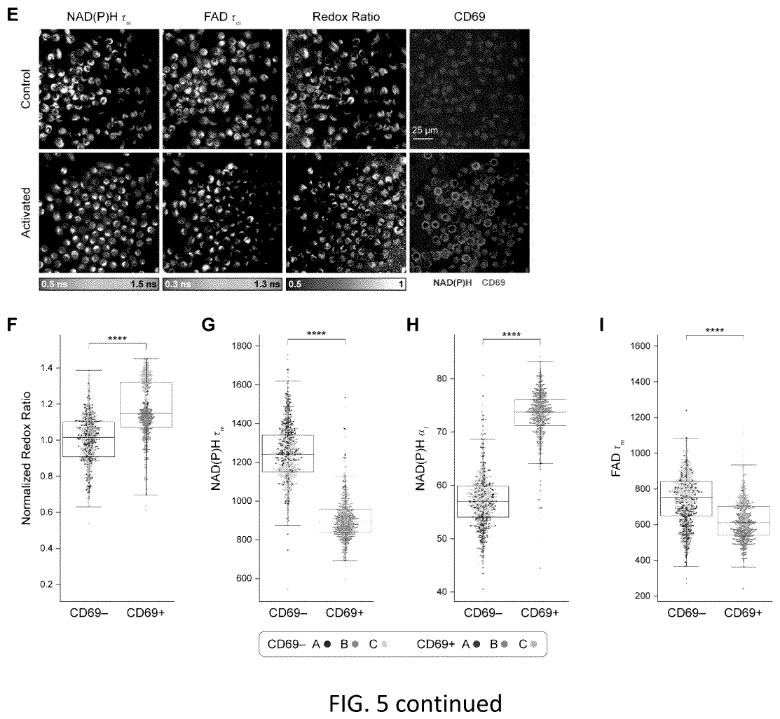

A graphical overview of the experiment is provided in FIG. 5A. Isolated human B cells were activated using anti-CD40 antibody and IL-4 to mimic T cell mediated activation. After 72 hours of in vitro activation, media was collected for cytokine, glucose, and lactate assays, then cells were stained with anti-CD69 PerCP antibody to identify activated and quiescent cells in each condition for subsequent OMI. To confirm that our protocol successfully activated the B cells, the concentration of IL-6 in the media was measured at 72 hours and found to significantly increase in the activated compared to the control condition (FIG. 5B). Similarly, analysis of glucose and lactate levels at 72 hours show decreased glucose and increased lactate in the media of activated compared to control B cells (FIG. 5C-D), confirming known metabolic changes with B cell activation. Representative images from OMI (FIG. 5E) include NAD(P)H mean fluorescence lifetime (τₘ), FAD τₘ, optical redox ratio, and PerCP-CD69 fluorescence images in pseudocolor. Qualitatively, most B cells in the activated condition stain positive for CD69.

The optical redox ratio was elevated in CD69+ B cells in the activated condition compared to CD69− B cells in the control condition (FIG. 5F). Additionally, NAD(P)H τₘ decreased and NAD(P)H α₁ (the fraction of free, unbound NAD(P)H) increased in CD69+ activated B cells compared to the CD69− control cells (FIG. 5G, 5H). FAD τₘ also decreased in the CD69+ activated cells compared to the CD69− control cells (FIG. 51).

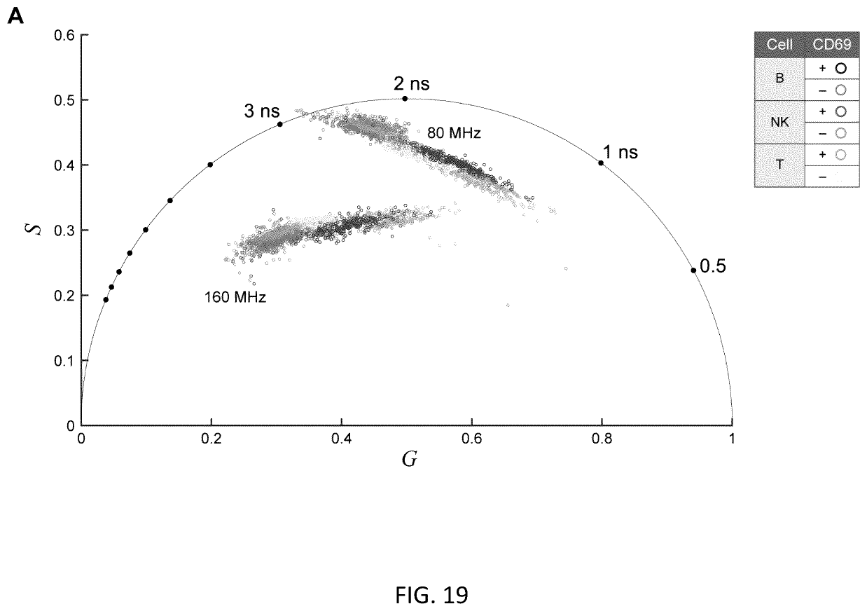

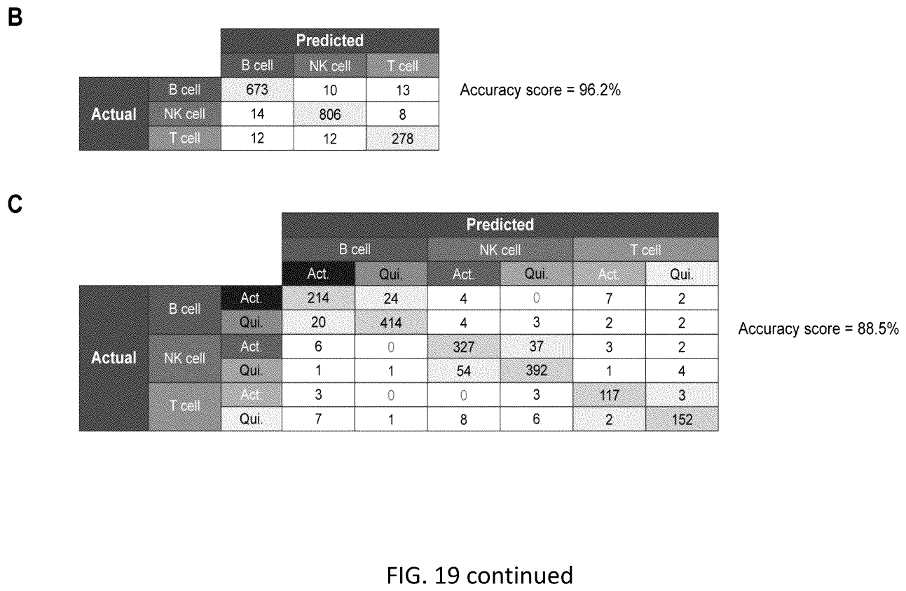

When comparing CD69+ and CD69− cells within the unstimulated or activated conditions, the CD69+ and CD69− B cells in the unstimulated condition did not show any significant differences in OMI parameters. However, in the activated condition, CD69+ cells were significantly different compared to CD69− cells for all OMI parameters besides the optical redox ratio (FIG. 19).

### Single Cell Clustering and Machine Learning Models Based on OMI Separate B Cells by Activation State

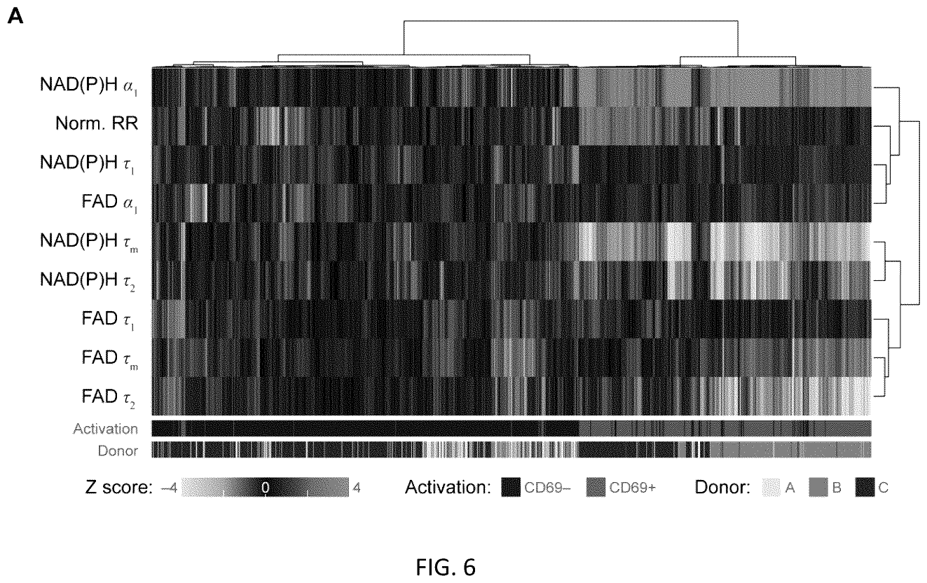

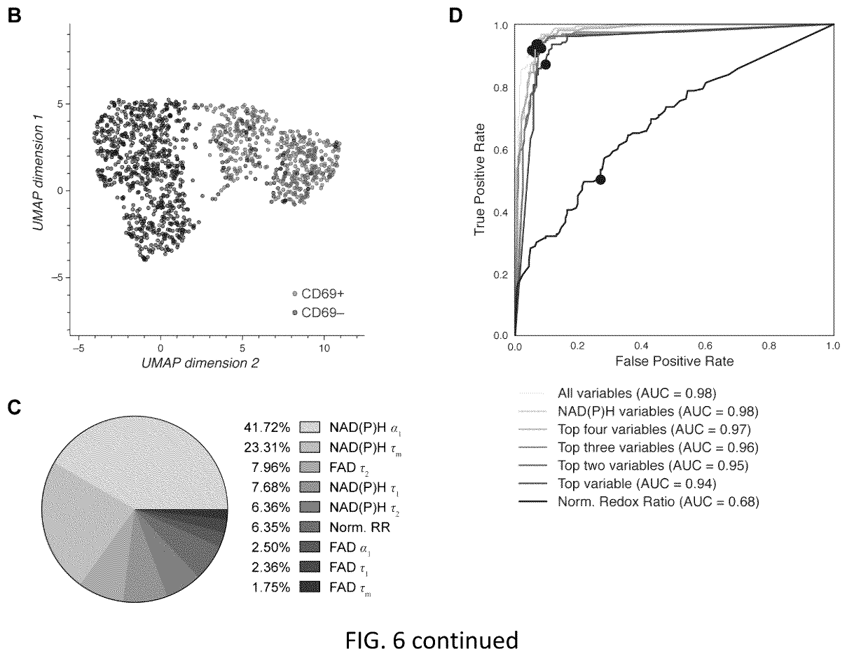

Next, we investigated whether OMI could visualize single cell heterogeneity in B cells and whether machine learning models based on OMI can classify B cell activation state. Unsupervised clustering of 9 OMI parameters from single cells in the CD69+ activated condition and CD69− control condition revealed that the CD69+ activated cells cluster separately from the CD69− control cells across all three donors (FIG. 6A). Uniform manifold approximation and projection (UMAP) was used to visualize the clustering of single B cells based on the same OMI parameters, which similarly revealed distinct clusters of CD69+ activated and CD69− control cells (FIG. 6B). Additional UMAPs colored by donor, condition (control, stimulated), and CD69 status are provided in FIG. 6A-B.

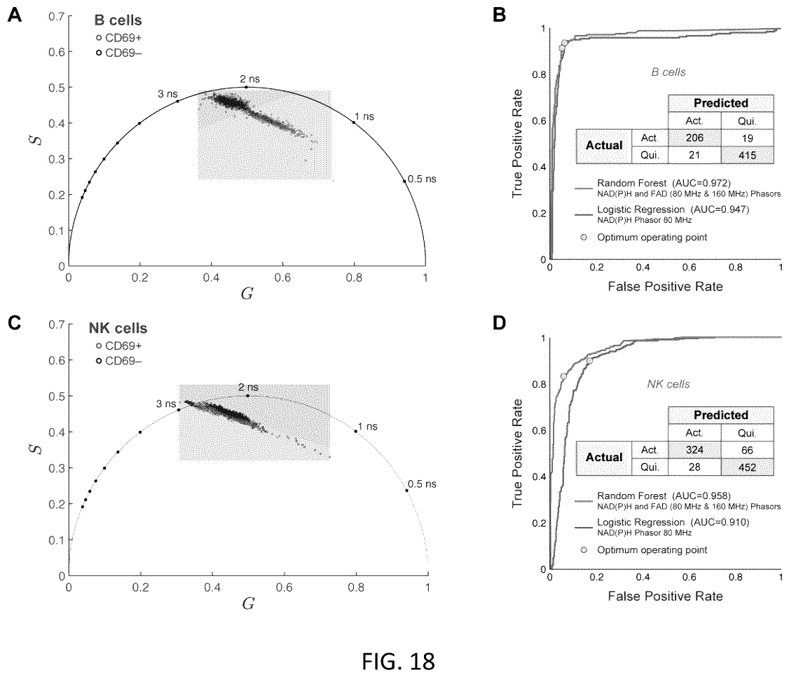

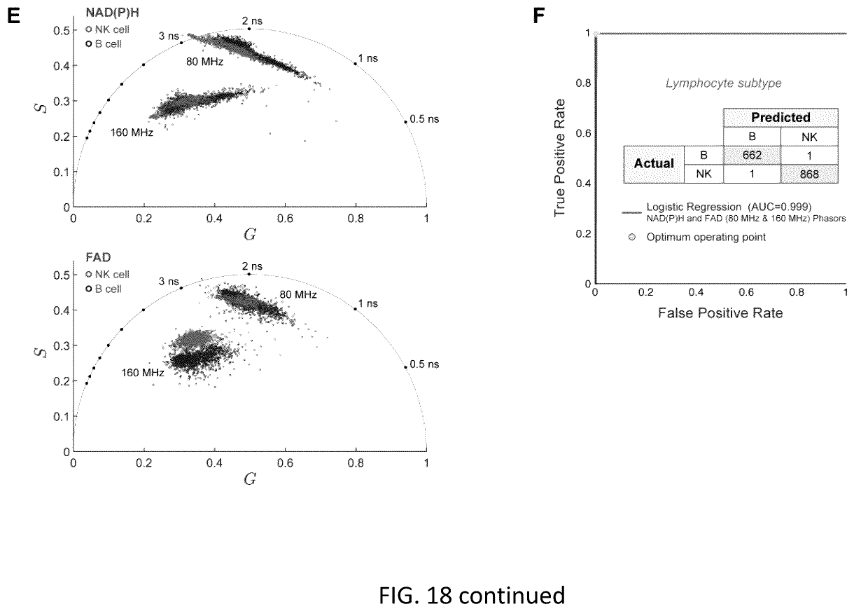

Next, a random forest classifier based on OMI parameters for each B cell was trained on 70% of the cells and tested on the remaining 30% of cells to identify activated (CD69+ in activated condition) or quiescent (CD69− in unstimulated condition) B cells. The OMI parameters with the greatest weight in the classification of CD69+ and CD69− B cells were NAD(P)H α₁ (40.33%), NAD(P)H τₘ (21.01%), FAD protein-bound fluorescence lifetime (n) (9.88%), and NAD(P)H free fluorescence lifetime (n) (7.46%) (FIG. 6C). The resulting classifier has an accuracy of 94.2% (FIG. 6C), with a receiver operating characteristic (ROC) area under the curve (AUC) of 0.99 (FIG. 6D). Logistic regression and support vector machine (SVM) classification performed similarly to the random forest classifier (FIG. 6C-F). Classification based on the NAD(P)H phasor at the laser repetition frequency (80 MHz) predicted B cell activation with 93.4% accuracy (FIG. 18A-B).

### OMI Resolves Metabolic Differences Between Quiescent and Activated Human NK Cells

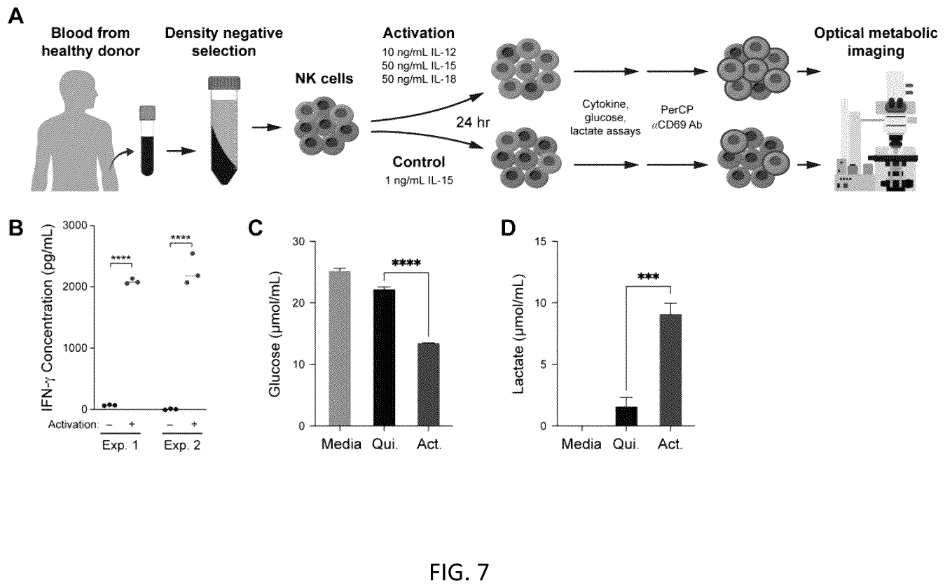

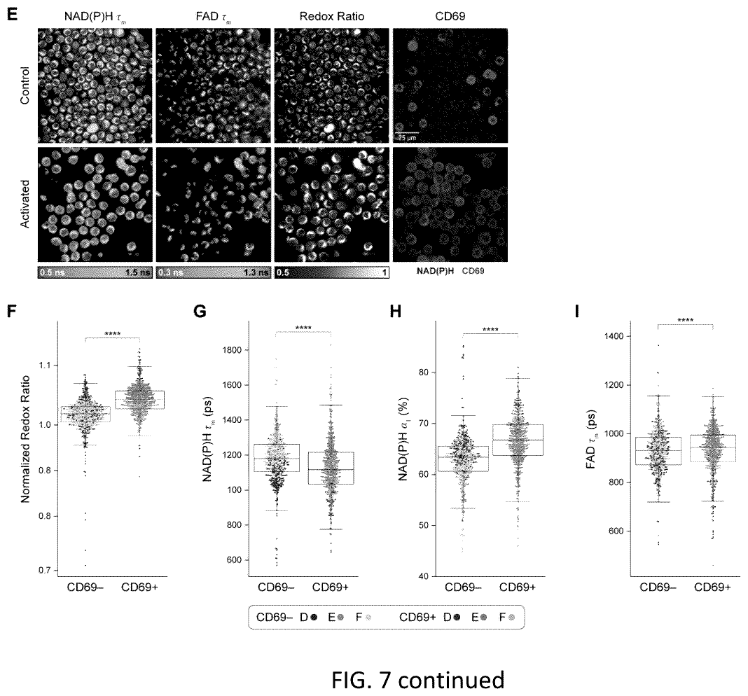

A graphical overview of the experiment is provided in FIG. 7. Isolated primary human NK cells were activated in vitro for 24 hours using IL-12, IL-15, and IL-18 as previously described. After 24 hours of in vitro activation, media was collected for cytokine, glucose, and lactate assays, then cells were stained with anti-CD69 PerCP antibody to identify activated and quiescent cells in each condition for subsequent OMI. To confirm NK cell activation, the concentration of IFN-γ in the media was measured at 24 hours and found to significantly increase in the activated compared to the control condition (FIG. 7B). Similarly, analysis of glucose and lactate levels at 24 hours show decreased glucose and increased lactate in the media of activated compared to control NK cells (FIG. 7C-D), confirming known metabolic changes with NK cell activation. Representative images of NAD(P)H τₘ, FAD τₘ, optical redox ratio, and PerCP-CD69 are presented in pseudocolor (FIG. 7E). Qualitatively, most NK cells in the activated condition stain positive for CD69.

OMI of NK cells revealed several changes in the CD69+ NK cells in the activated condition compared to CD69− cells in the control condition. The optical redox ratio significantly increased in the CD69+ activated NK cells compared to CD69− control cells (FIG. 7F). NAD(P)H τₘ decreased, and NAD(P)H α₁ and FAD τₘ increased in activated NK cells compared to the quiescent control cells (FIG. 7G-I).

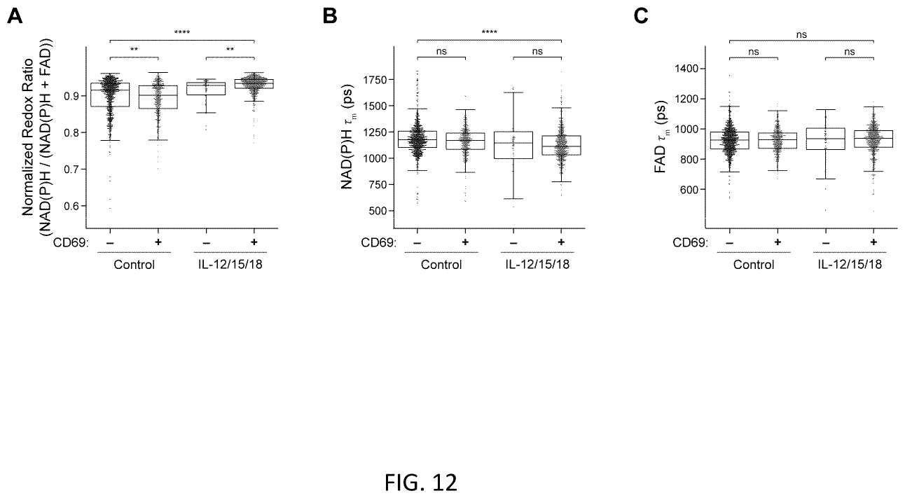

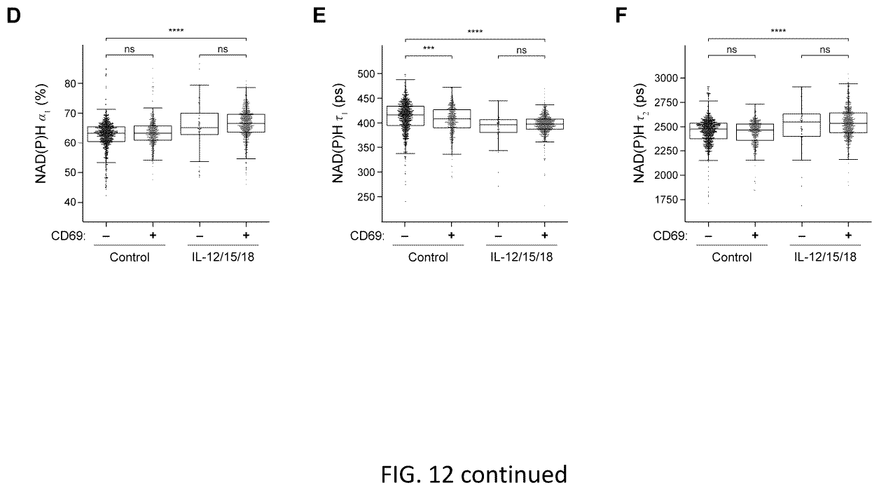

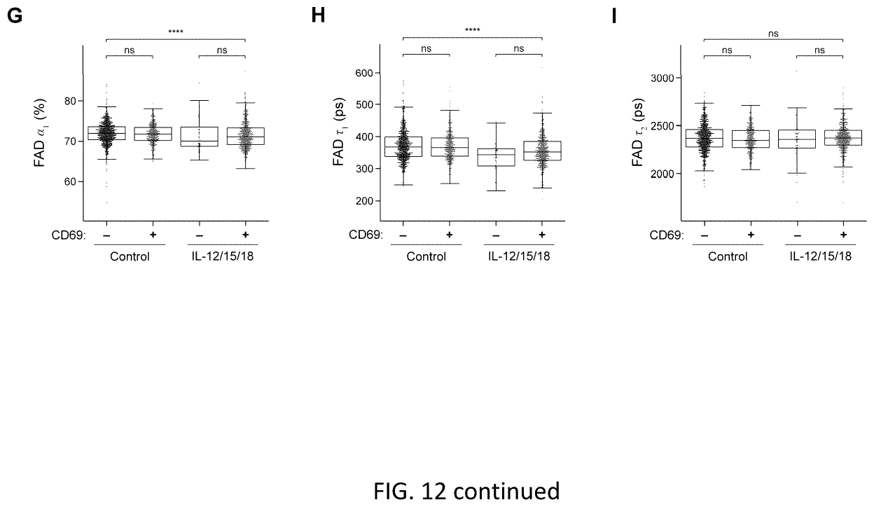

OMI parameters were compared across both CD69+ and CD69− NK cells in the activated and control conditions. Most OMI parameters did not change with CD69 status within the activated or control conditions, besides the optical redox ratio (control and activated conditions) and NAD(P)H τ₁ (control condition) (FIG. 12).

### Single Cell Clustering and Machine Learning Models Based on OMI Separate NK Cells by Activation State

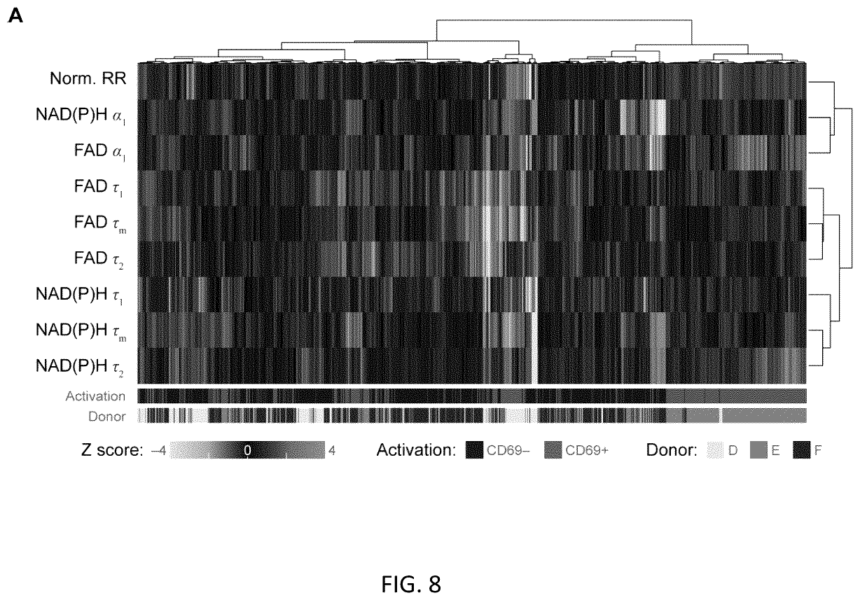

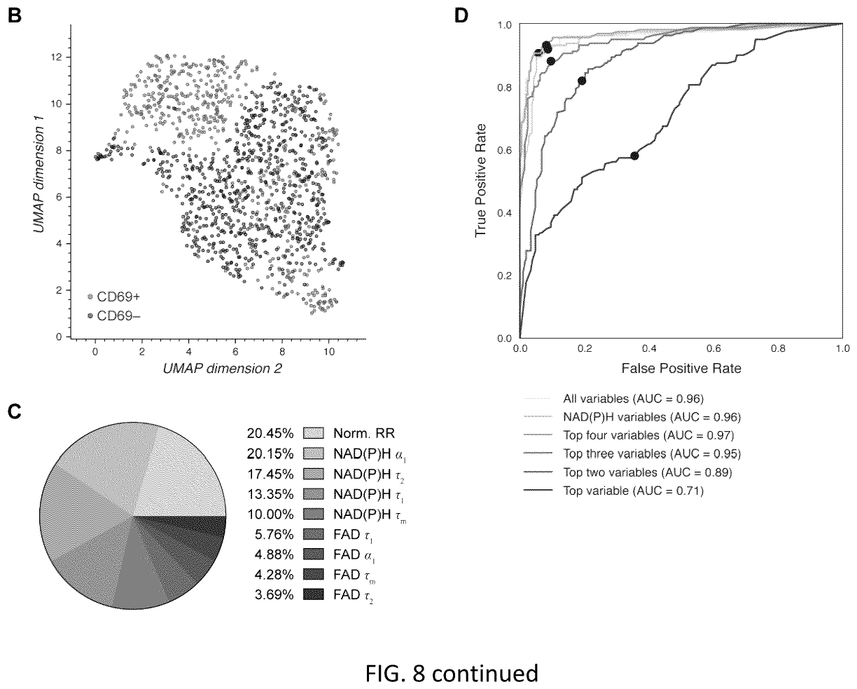

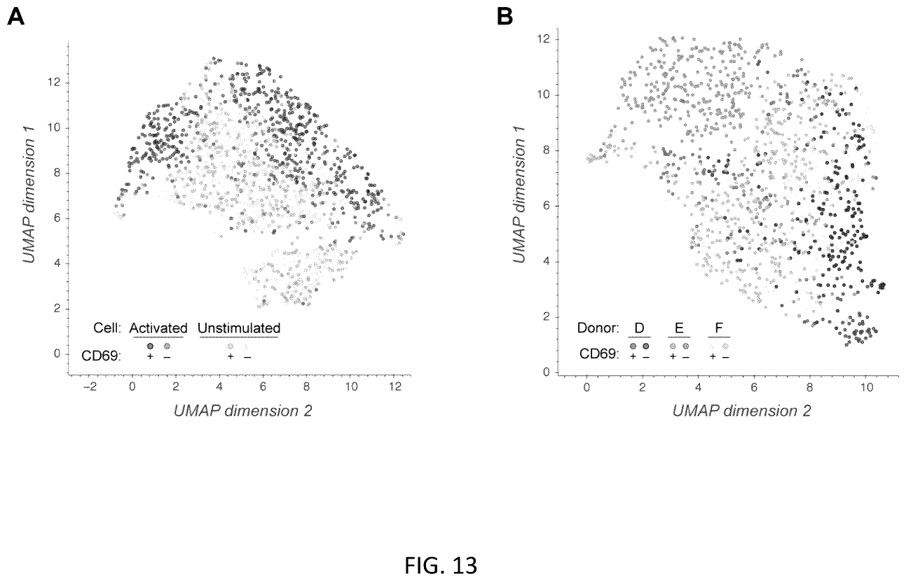

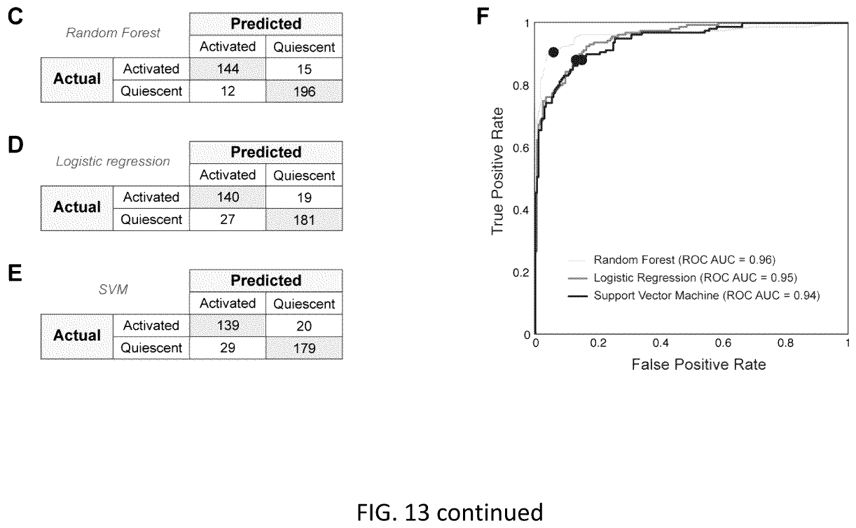

Next, we investigated whether OMI could visualize single cell heterogeneity in NK cells and whether machine learning models based on OMI can classify NK cell activation state. Unsupervised clustering of 9 OMI parameters from single cells in the CD69⁺ activated condition and CD69⁻ control condition revealed that NK cells were somewhat heterogeneous, resulting in the emergence of a dominant cluster with several smaller clusters of activated and quiescent cells (FIG. 4A). A UMAP was used to visualize the clustering of single NK cells based on the same OMI parameters, which demonstrated a cluster of CD69+ NK cells away from a cluster of a mixed CD69− and CD69+ NK cell population (FIG. 8B). Further color-coding by donor reveals that NK cells from all three donors overlap in these clusters (FIG. 13B).

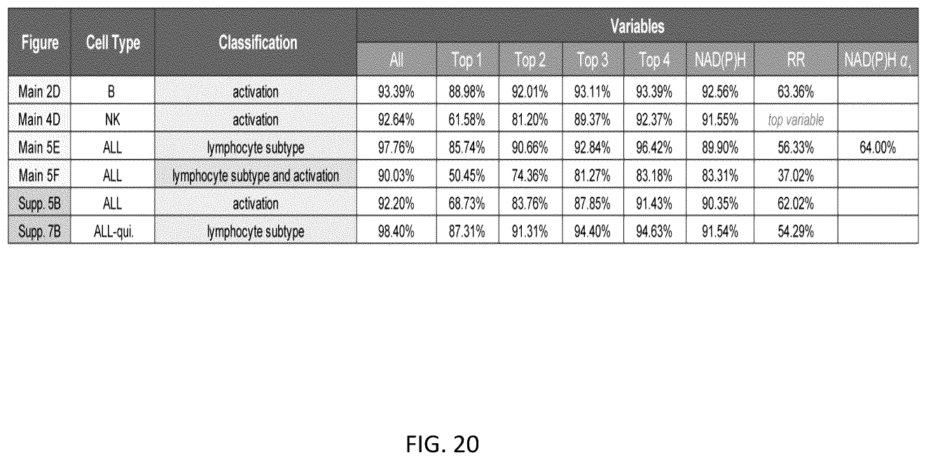

A random forest classifier based on single-cell OMI parameters was trained and tested on 70% and 30%, respectively, of the NK cells to identify activated (CD69+ in activating conditions) or quiescent (CD69− in unstimulated conditions) states. The highest weighted OMI parameters were the control-normalized optical redox ratio (20.45%), NAD(P)H α₁ (20.15%), protein-bound NAD(P)H fluorescence lifetime (τ₂) (17.45%), and unbound NAD(P)H fluorescence lifetime (τ₁) (13.35%) (FIG. 8C). The resulting classifier had an accuracy of 92.6% (FIG. 13C, FIG. 20 FIG. 20), and the AUC of the ROC curve was 0.96 (FIG. 8D). Logistic regression and SVM classification had a slightly lower performance than random forest classification, with AUC of the ROC curves of 0.95 and 0.94, respectively (FIG. 8C-F). Classification based on the NAD(P)H and FAD phasors at both the laser repetition frequency (80 MHz) and its second harmonic (160 MHz) predicted NK cell activation with 89.2% accuracy (FIG. 18C-D).

### OMI Quantifies Lymphocyte Heterogeneity and Classifies Lymphocyte Subtype and Activation State

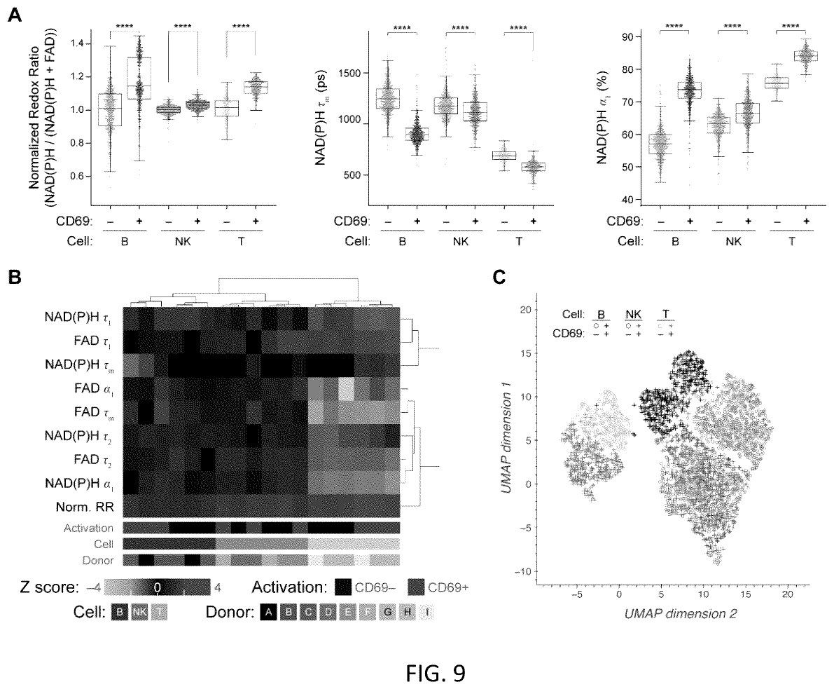

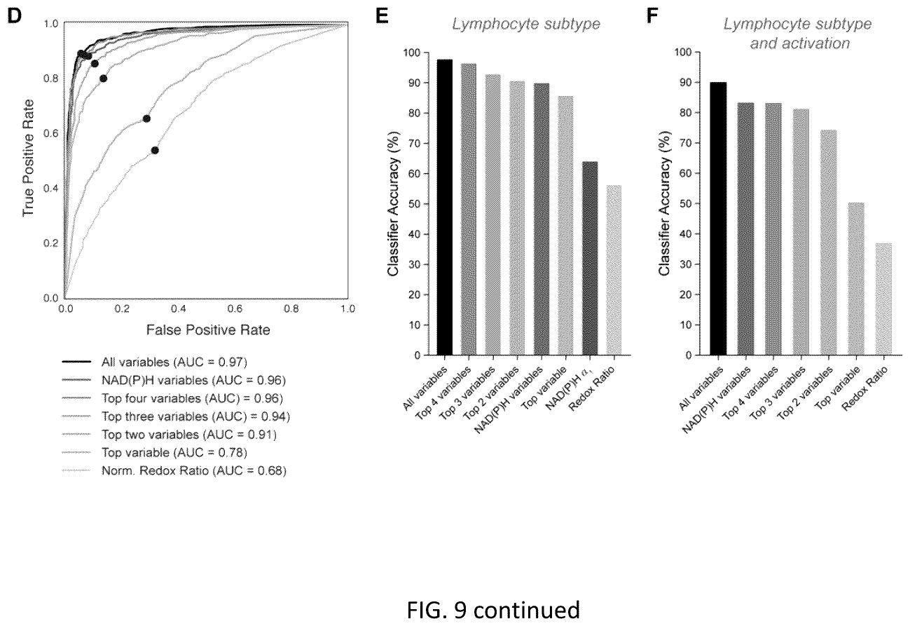

Whether OMI parameters could distinguish activation and/or lymphocyte subtype across a dataset containing multiple subtypes of lymphocytes was investigated. We combined the NK cell and B cell data with our previously published T cell data (quiescent and activated for 48 h with CD2/3/28) and plotted several key OMI parameters, including the control-normalized optical redox ratio, NAD(P)H τₘ, and NAD(P)H α₁ (FIG. 9A). Across all three lymphocyte subtypes, these variables exhibited similar changes with activation: NAD(P)H τₘ decreased with activation, while NAD(P)H α₁ and the optical redox ratio increased with activation. These changes with activation were statistically significant in all cases. In addition to activation-associated shifts in OMI parameters, there were also statistically significant differences between quiescent T, B, and NK cells (FIG. 9A).

The combined data set of T, B, and NK cells was used to visualize heterogeneity between each group. Unsupervised clustering was performed across 9 OMI parameters using averages from CD69⁺ activated and CD69⁻ control lymphocytes across activation state (CD69+, CD69−), donor, and lymphocyte subtype (B, NK, T cell) (FIG. 9B). This revealed distinct clusters of CD69− and CD69+ T cell and B cell groups. Within the NK cells, clustering was mixed across CD69− and CD69+ status and donors. A UMAP was also used to further visualize clustering, with T cells forming a distinct cluster from B cells and NK cells (FIG. 9C).

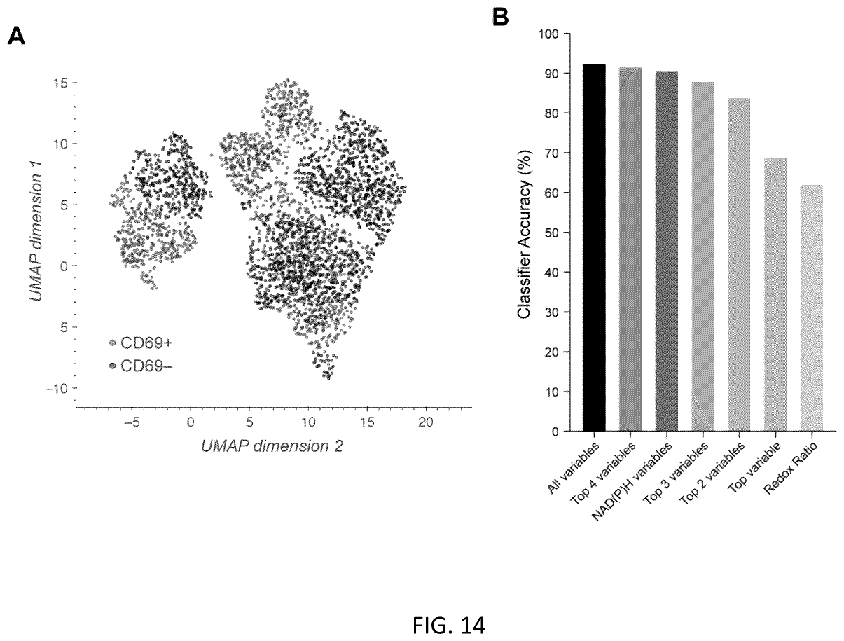

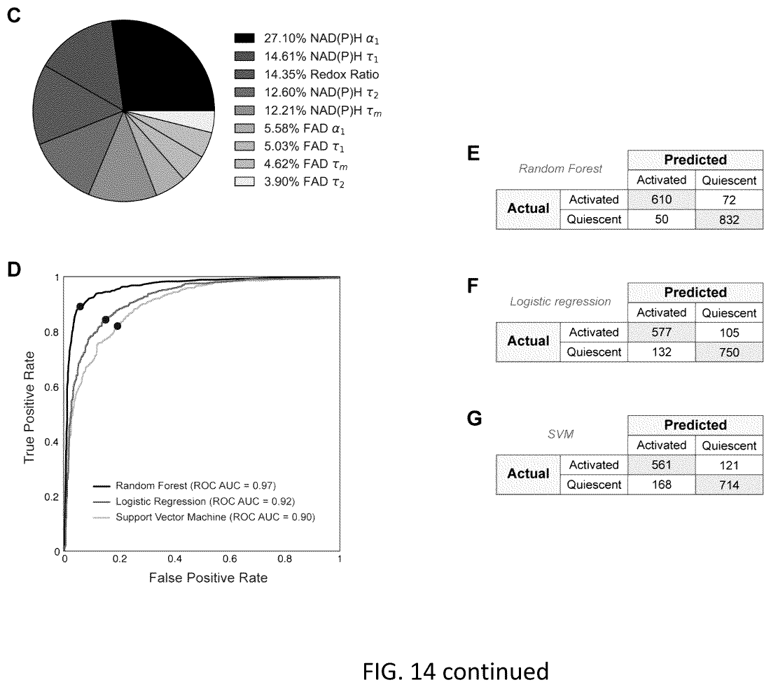

A UMAP reveals that CD69+ lymphocytes clustered somewhat separately from CD69− lymphocytes (FIG. 14A). Therefore, we investigated whether machine learning models could classify activation within the combined lymphocyte data. First, random forest classification was used to identify whether cells were activated (CD69+) or quiescent (CD69−). Using all 9 OMI parameters, an ROC AUC of 0.97 (FIG. 9D) and accuracy of 92.2% (FIG. 14B, FIG. 20) was achieved. The top feature weights were NAD(P)H α₁ (27.10%), NAD(P)H τ₁ (14.61%), control-normalized optical redox ratio (14.35%), and NAD(P)H τ₂ (12.60%) (FIG. 13C). Classification based on NAD(P)H lifetime variables (τₘ, τ₁, τ₂, α₁) alone also had a high ROC AUC of 0.96 (FIG. 5D) and performance (accuracy=90.3%) (FIG. 14B, FIG. 20). Logistic regression and support vector machine classification performance were somewhat diminished from the random forest classification performance, with accuracies of 84.8% and 81.5% respectively (FIG. 14D-G).

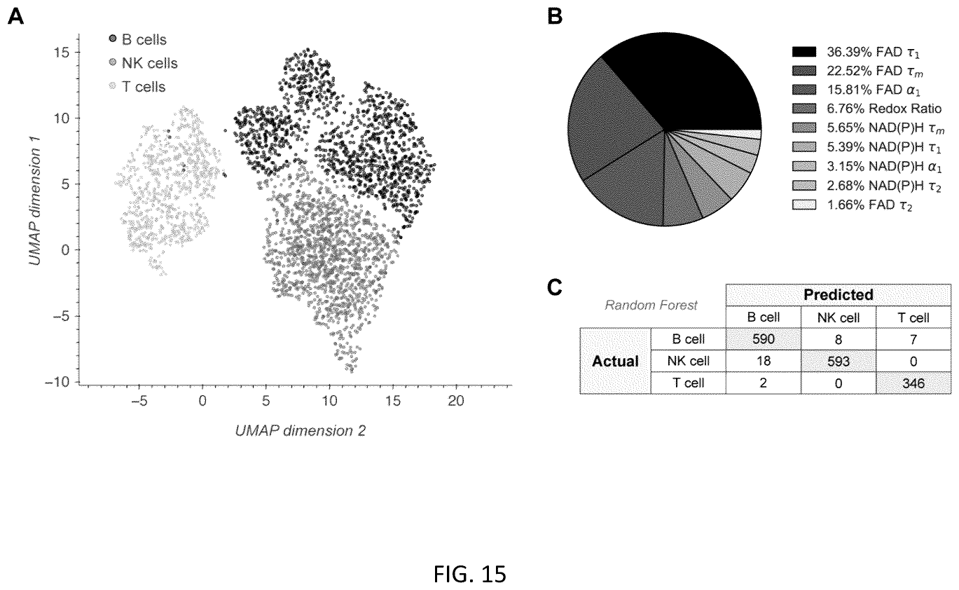

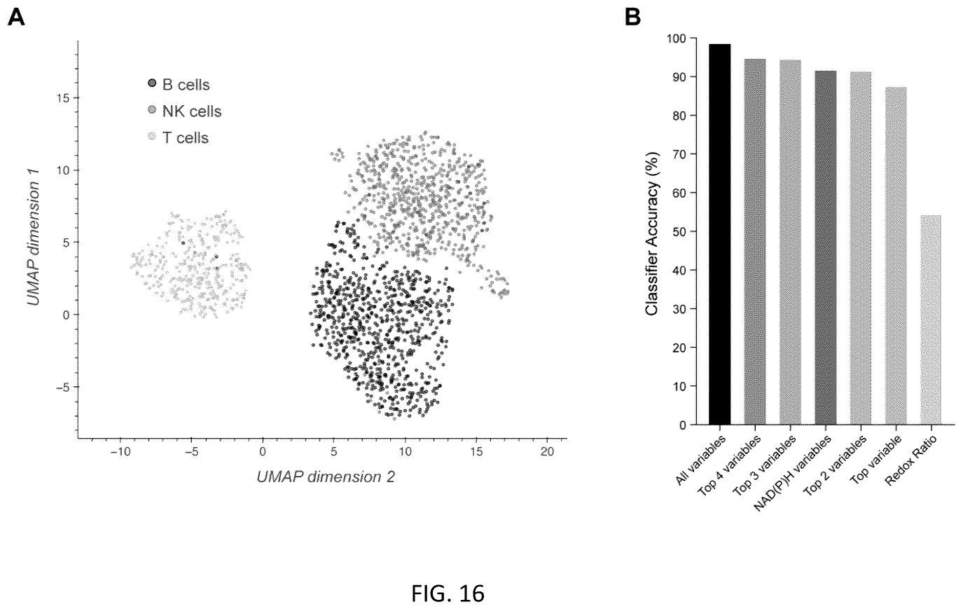

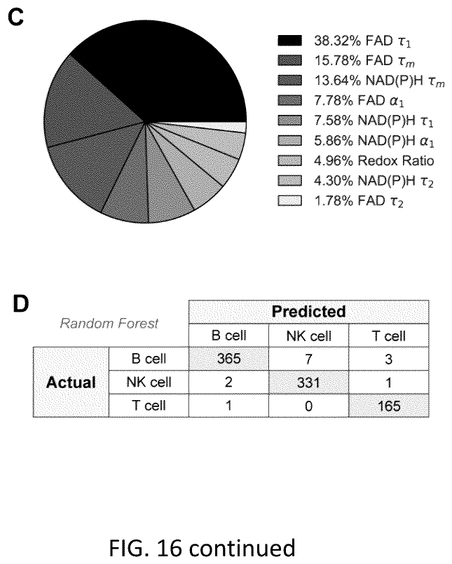

Similarly, a UMAP reveals that T cells, B cells, and NK cells clustered separately (FIG. 15A). Therefore, three-class random forest classification of lymphocyte subtype was then performed (one vs. one approach) using different combinations of OMI parameters. Feature weights are provided (FIG. 15B). Classification with all nine OMI parameters performed the best (accuracy=97.8%) (FIG. 9E, FIG. 14C, FIG. 20). However, other random forest classifiers with fewer parameters also demonstrated strong performance. The top four parameters (FAD τᵢ, FAD τₘ, NAD(P)H τₘ, and FAD α₁) had an accuracy of 96.4%, while NAD(P)H lifetime variables (τₘ, τ₁, τ₂, α₁) had an accuracy of 89.9% (FIG. 9E, FIG. 20 FIG. 20). Classification using both NAD(P)H and FAD 80 MHz and 160 MHz phasors classified cells as either B or NK cells with 99.9% accuracy (FIG. 18E-F), while the NAD(P)H 80 MHz and 160 MHz phasor classified cells as B, NK, or T cells with 96.2% accuracy (FIG. 19A-B). Classification was also tested on a subset of the combined lymphocyte data that contained only quiescent (CD69−) cells (UMAP, FIG. 16A), and had similarly high performance with all nine OMI parameters (accuracy=98.4%) (FIG. 16B-D, FIG. 20).

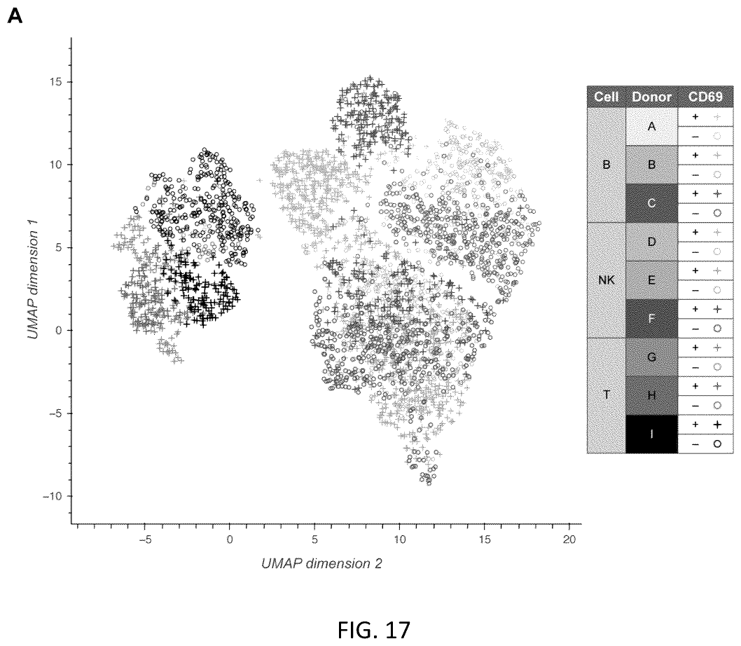

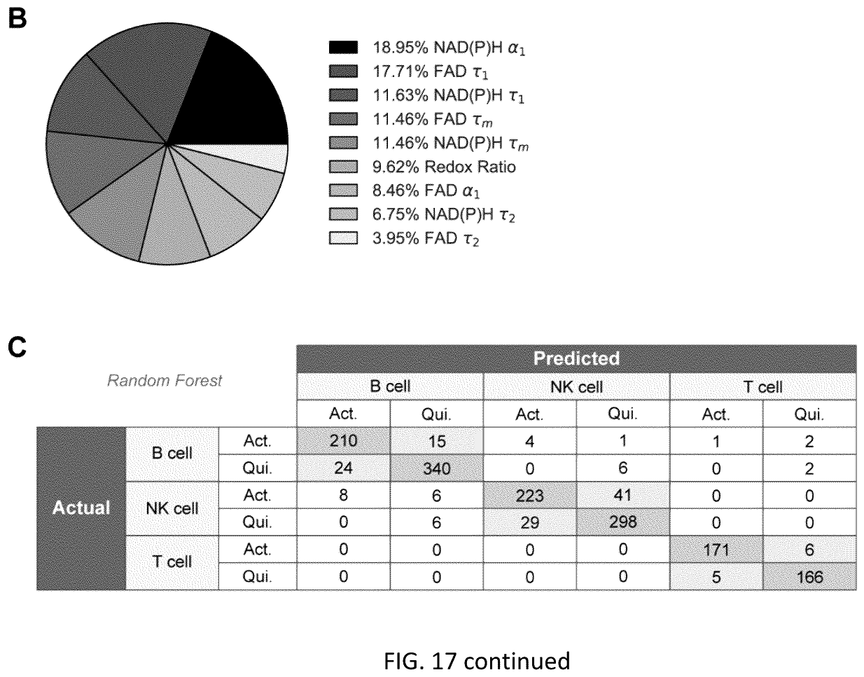

Finally, a UMAP shows that quiescent and activated T cells, B cells, and NK cells clustered separately (FIG. 17A). Therefore, random forest classification was used to classify both lymphocyte subtype and activation simultaneously. A six-class classification was performed (one vs. one approach). Feature weights are provided (FIG. 17B). Again, the classifier with all 9 OMI parameters had the highest accuracy (accuracy=90.0%) (FIG. 9F, FIG. 20), and misclassification was highest between quiescent vs. activated cells within a lymphocyte subtype, with lymphocyte subtype usually identified correctly (FIG. 17C). Other classifiers also performed well, including the top four parameters (NAD(P)H α₁, FAD τ₁, NAD(P)H τ₁, FAD τₘ) with an accuracy of 83.2%, and NAD(P)H lifetime variables (τₘ, τ₁, τ₂, α₁) with an accuracy of 83.3% (FIG. 9F, FIG. 20). Classification using NAD(P)H 80 MHz and 160 MHz phasor classified both lymphocyte subtype and activation simultaneously with an accuracy of 88.5% (FIG. 19C). A summary of the accuracies of all random forest classifiers is provided (FIG. 20).

### Discussion

Several areas of research and clinical care rely on lymphocyte assessments, but these efforts would benefit from a non-destructive, single-cell, touch-free technology to assess lymphocyte subtype and activation state, which would reduce the cost and time for analysis of heterogeneity within a patient while enabling subsequent study and use of unperturbed cells. In this report, we have demonstrated that OMI is sensitive to metabolic changes that occur with activation in primary human B cells and NK cells. Additionally, machine learning models trained on single-cell OMI parameters can reliably classify quiescent cells in both CD40/IL4 activated B cells and IL12/IL15/IL18 activated memory-like NK cells, as well as distinguish lymphocyte subtypes (NK, B, T cells) and activation within a combined dataset of NK, B, and T cells.

Interestingly, both B cells and NK cells had similar changes in OMI parameters under activation compared to quiescent cells. The optical redox ratio increased, NAD(P)H τₘ decreased, and NAD(P)H α₁ increased in the activated cells (FIGS. 5, 7). These trends are consistent with our prior work with primary human T cell activation 47. The similarity of changes between the B cells, NK cells, and T cells is likely related to similar shifts in metabolism when all three lymphocyte subtypes are activated. All three types of lymphocytes upregulate oxidative phosphorylation and aerobic glycolysis when activated to fuel rapid growth and proliferation.

Previous studies have demonstrated that alterations to glycolysis significantly affect OMI measurements. Specifically, inhibition of glycolysis with 2DG selectively reduced the optical redox ratio in activated T cells, indicating that glycolysis is a key regulator of the optical redox ratio in these cells 47. Further, the optical redox ratio has a positive correlation (Pearson's R=0.89) with the glycolytic index of breast cancer cells. In this study, measurements of glucose and lactate levels in media from control and activated B and NK cells revealed that the glucose concentration significantly decreased and the lactate concentration significantly increased with activation (FIGS. 5, 7). This observation is consistent with upregulation of aerobic glycolysis noted in the literature, as well as our observed increase in the optical redox ratio with activation.

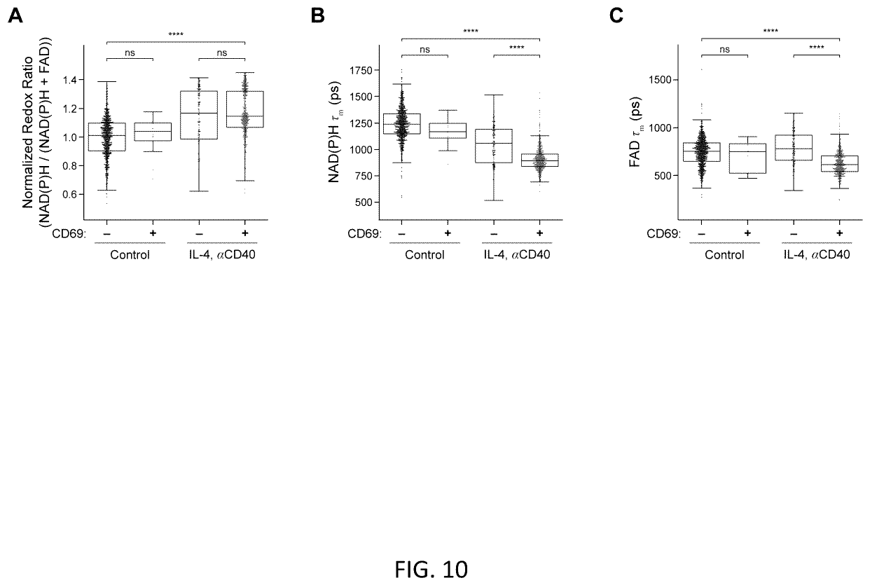

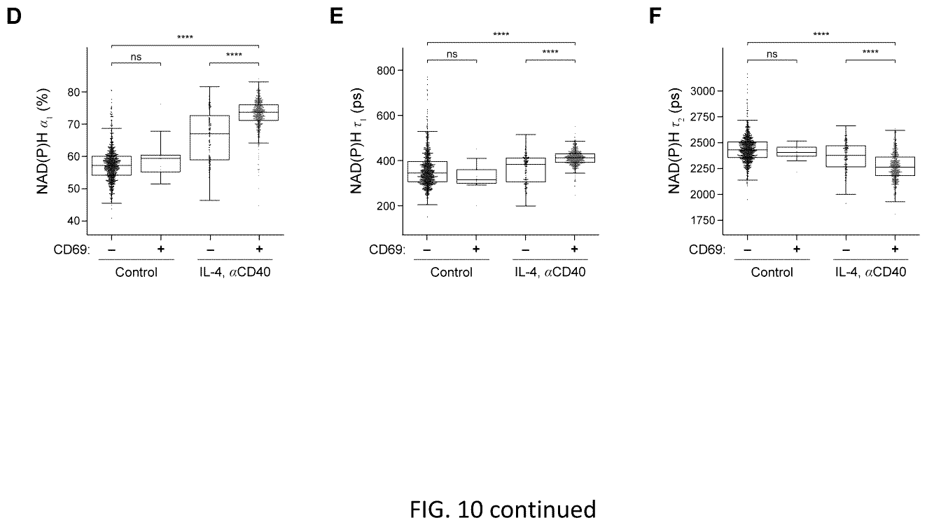

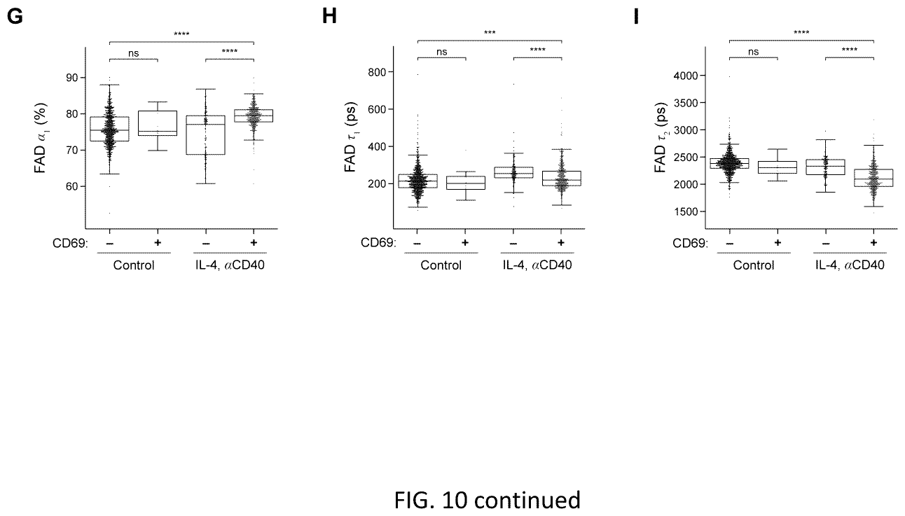

The single-cell resolution of OMI makes it a powerful tool for studying and characterizing population heterogeneity. Here, we characterized heterogeneity within activated and quiescent B cell and NK cell populations. Our results demonstrate that within a population of peripheral human B cells or NK cells exposed to the same conditions, cell outcomes may vary. Examination of CD69 expression revealed that there was a mixture of CD69+ and CD69− cells within each group despite exposure to the same media conditions. We chose to focus the analysis on cells that we could confirm to be quiescent (i.e., CD69− cells in the quiescent condition) and cells that we could confirm to be activated (i.e., CD69+ cells in the activated condition) to better characterize the ability of OMI to assess these cells without complications that could arise from differing cell states. However, OMI did capture differences in NAD(P)H and FAD autofluorescence between activated and quiescent cells within each condition (FIGS. 10, 12). Overall, this study demonstrates the sensitivity of OMI to metabolic differences within a heterogenous cell population.

Classifiers based on single-cell OMI accurately identified activation state with up to 93.4% accuracy for B cells and up to 92.6% accuracy for NK cells (FIG. 20). In addition to classifying activation within a lymphocyte subtype, OMI also classified activation with high accuracy from a combined dataset of T cells, B cells, and NK cells (accuracy=92.2%, 9 OMI parameters) (FIG. 14B, 14E, FIG. 20). We also found that all 9 OMI parameters accurately classified lymphocyte subtype (accuracy of 97.8%, FIG. 9E, FIG. 15C, FIG. 20), which reflects the distinct fluorescence lifetimes of NAD(P)H and FAD for different lymphocyte subtypes (FIG. 9A). Indeed, a previous study has shown that NAD(P)H and FAD autofluorescence differs between different types of murine white blood cells (including B cells and T cell subtypes). OMI parameters also distinguished between lymphocyte subtypes when only quiescent (CD69− control) cells were used (accuracy of 98.4%, FIG. 16B, FIG. 20). Differences in NAD(P)H and FAD fluorescence lifetimes between quiescent lymphocytes may be explained by differences in resting cell metabolism between T cells, B cells, and NK cells, which has been observed in previous human and murine studies. Surprisingly, even a six-group classifier of both activation state and lymphocyte subtype achieved high accuracy (90.0%, FIG. 9F, FIG. 17C, FIG. 20) which reflects subtle changes in metabolic state for these six classes.

Although a complete set of NAD(P)H and FAD intensities and lifetimes were collected in this study, all 9 OMI parameters may not be necessary for accurate classification. NAD(P)H lifetime variables alone accurately classified activation within B cells (92.6% accuracy), activation within NK cells (91.6%), activation within the combined dataset of B cells, NK cells, and T cells (90.3% accuracy), lymphocyte subtype (89.9% accuracy), and a six-class classifier of both activation and lymphocyte subtype (83.3% accuracy) (FIG. 20), while the NAD(P)H phasor alone classified B cell activation and NK cell activation with accuracies of 93.9% and 88.1% respectively (FIG. 18A-D), lymphocyte subtype with 96.2% accuracy (FIG. 19A-B), and both activation and lymphocyte subtype with an accuracy of 88.5% (FIG. 19C). This indicates that simplified hardware with only NAD(P)H excitation and emission capabilities would perform as accurately as a two-color NAD(P)H and FAD imaging system, which is an important consideration in the design of simplified hardware for use in clinical labs.

Overall, these studies indicate that OMI can robustly classify lymphocyte activation status and discriminate B cells, NK cells, and T cells. This label-free single-cell imaging and classification approach could have significant implications in cell manufacturing, where in-line technologies are needed to maintain high potency and safety, or in clinical labs where immune cell profiling is needed to inform treatment decisions. The non-invasive nature of this approach also enables time-course studies of lymphocyte function and in vivo studies of lymphocytes in a native context.

### Limitations of Study

Cells must remain viable (i.e., no fixation) for this method to succeed. Unlike flow cytometry, OMI does not provide surface marker expression or traditional phenotyping based on surface markers. Therefore, OMI is not appropriate for studies where high depth of molecular information is needed. High-content single-cell analysis is better performed with flow cytometry, CyTOF, and/or single-cell RNA sequencing. However, OMI is advantageous when touch-free, non-invasive, and rapid measurements are beneficial, such as continuous monitoring within unperturbed systems (cell culture, 3D culture, in vivo), cell therapy production where good manufacturing practice (GMP) must be maintained to generate cells for patient use, and when rapid reactivity tests are needed (e.g., immune profiling).

Data and materials availability: All data and code used in the analyses is available for purposes of reproducing or extending the analyses through a GitHub repository (https://github.com/skalalab/schmitz_r-lymphocyte_activation).

### Materials and Methods

### Isolation of Primary Human Lymphocytes

Primary human lymphocytes were isolated from peripheral blood obtained from healthy adult donors under approval by the UW-Madison Institutional Review Board. After obtaining informed consent from the donors, 10 to 50 mL whole blood was drawn using a sterile syringe with heparin. B cells and NK cells were then isolated from donor whole blood using negative isolation kits.

For NK cells, blood was mixed in a 1:1 ratio with 1×PBS. The peripheral blood:PBS mixture was then overlayed dropwise onto 15 mL of Lymphoprep (STEMCELL Technologies) in 50 mL conical tubes and centrifuged at 400×g for 30 minutes at 20° C. with slow acceleration and no breaks. After centrifugation, the PBMC layer was moved to a fresh 50 mL conical tube using a 10 mL serological pipette, with 35 mL 1×PBS added to each tube. Cells were centrifuged at 400×g for 10 min at 20° C. with normal acceleration and breaks. After centrifugation supernatant was aspirated and cell pellets were resuspended in 5 mL of ACK lysing buffer (Quality Biological) and let sit at room temperature for 5 minutes. ACK lysis reaction was then quenched with 30 mL of 1×PBS per 50 mL tube. Cells were centrifuged at 400×g for 10 min at 20° C. with normal acceleration and breaks. Supernatant was aspirated, with the pellets being combined in 40 mL 1×PBS and passed through a 70 μm filter. PBMCs were counted on the Z1 Particle Counter (Beckman Coulter) by adding 10 μL of the PBMC solution to 10 mL of Isoton II diluent (Beckman Coulter) in a 20 mL cuvette. PBMCs were then labelled with the human NK Cell Isolation Kit (Miltenyi Biotec), with subsequent NK cell isolation using the “depletes” program on an autoMACS Pro Separator and collecting the negative fraction. The isolated cells were then transferred to a cell culture flask or well plate for culture.

For the B cell isolation (EasySep, STEMCELL Technologies), peripheral blood mononuclear cells (PBMCs) were first isolated by diluting the blood with an equal volume of DPBS+2% FBS, then centrifuging at 1200×g for 10 minutes in SepMate tubes containing a layer of Lymphoprep. The isolated PBMCs were then washed with DPBS+2% FBS and centrifuged at 100×g for 10 minutes. The resulting sample was resuspended to a concentration of 50 million cells/mL in EasySep Buffer (STEMCELL Technologies). 50 μL/mL isolation cocktail and 50 μL/mL cocktail enhancer were added to the sample, according to the EasySep protocol. 50 μL/mL RapidSpheres solution was then added, and the sample was transferred to a magnet for 3 minutes. The enriched B cells were poured into a new tube and the sample was again placed into a magnet for 1 minute. The enriched B cell population was then washed with culture medium and transferred to a cell culture flask or well plate for culture.

### Lymphocyte Activation and Culture

NK cells were cultured in TheraPeak X-VIVO-10 medium (Lonza) supplemented with 10% human serum AB (Sigma Aldrich) and 1 ng/mL IL-15 (Biolegend). B cells were cultured in RPMI containing 5% fetal bovine serum and 1% penicillin-streptomycin. Following isolation, each cell population was divided into two groups: a control population cultured in normal medium, and an activated population cultured in control medium supplemented with additional components. NK cell activating medium was supplemented with 10 ng/mL IL-12 (Invivogen), 50 ng/mL IL-15, and 50 ng/mL IL-18 (Biolegend). B cell activating medium was supplemented with 5 μg/mL anti-CD40 antibody (R&D systems) and 20 ng/ml IL-4 (R&D Systems).

The cells were cultured separately in activating or control medium for a number of hours depending on the lymphocyte subtype; B cells were activated for 72 hours, and NK cells for 24 hours. Cells were seeded at a density of 1 million cells/mL medium. At the end of the activation time, a sample of growth medium from each group was taken for cytokine analysis. A summary of the isolation and activation conditions used is provided in Table 1.

TABLE 1
Isolation and activation conditions for each lymphocyte subtype.

| Lymphocyte | Negative | | Activation | Activation |
|---|---|---|---|---|
| subtype | Isolation Kit | Control Medium | Medium | time |
| B cell | Easy Sep Human | RPMI + 5% FBS + 1% | Control medium + | 72 hours |
| | Naïve B Cell | penicillin/streptomycin | 5 μg/mL anti- | |
| | Isolation Kit | | CD40 antibody + | |
| | (StemCell | | 20 ng/mL IL-4 | |
| | Technologies) | | | |
| NK Cell | MACS Human | TheraPeak X-VIVO-10 | Control medium + | 24 hours |
| | NK Cell | medium (Lonza) + | 10 ng/ml IL-12 + | |
| | Isolation Kit | 10% human serum | 50 ng/ml IL-15 + | |
| | (Miltenyi Biotec) | AB + 1 ng/mL IL-15 | 50 ng/ml IL-18 | |

Staining with PerCP Conjugated Anti-CD69 Antibody

At the end of the activation period, cells were stained with anti-CD69 PerCP-conjugated antibody to distinguish activated and quiescent cells within each population. The cells were centrifuged at 300×g for 8 minutes, then resuspended to a concentration of 10 million cells/mL medium. 5 μL/million cells PerCP-conjugated anti human CD69 antibody (Biolegend) was added to the sample. The cells were then incubated for 30 minutes at room temperature. Following incubation, the cells were washed twice with media and centrifuged at 300×g for 8 minutes to remove excess antibody from the sample.

### Fluorescence Lifetime Imaging of Lymphocytes

For imaging, B cells and NK cells were plated 1 hour before imaging on poly-D-lysine coated glass-bottomed dishes (MatTek) at a seeding density of 200,000 cells in 50 μL media. The cells were imaged with a custom-built multiphoton fluorescence microscope (Ultima, Bruker) using a 100× (NA=1.45) oil immersion objective and time-correlated single photon counting electronics (SPC-150, Becker & Hickl GbH, Berlin, Germany). The laser (Insight DS+, Spectra-Physics Inc., Santa Clara, CA, USA) was tuned to 750 nm for NAD(P)H excitation, 890 nm for FAD excitation, and 980 nm or 1040 nm excitation for PerCP. Fluorescence emission was detected using a H7422PA-40 GaAsP photomultiplier tube (Hamamatsu Corporation, Bridgewater, NK, USA) and isolated using a 440/80 bandpass filter for NAD(P)H, 550/50 (NK cells) or 550/100 (B cells) bandpass filter for FAD, and 690/50 bandpass filter for PerCP. In the B cell experiments, the laser power at the sample was 1.5 mW-2.0 mW for NAD(P)H, 3.0 mW-4.0 mW for FAD, and 3.0 mW for PerCP. In the NK-cell experiments, the laser power at the sample was 2.0 mW for NAD(P)H, 5.0 mW for FAD, and 3.5 mW for PerCP. The laser power was maintained at a consistent value within each experiment.

300 μm×300 μm fluorescence lifetime images (256×256 pixels) were collected consecutively for NAD(P)H and FAD in the same field of view, with a pixel dwell time of 4.8 us and an integration time of 60 s. An instrument response function was collected during imaging from the second harmonic generation of a urea crystal, and photon count rates were maintained around 1×10⁵ photons. An intensity image of PerCP fluorescence was collected for the same field of view. Images were collected from three to six fields of view for each sample.

### Image Analysis

Fluorescence lifetimes were extracted through analysis of the fluorescence decay at each pixel in SPCImage (Becker & Hickl). To provide more robust calculations of the fluorescence lifetimes, a threshold was used to exclude background pixels with a low intensity, and images were binned up to a bin factor of 3 to reach a peak of at least 100 photons in the decay. Both NAD(P)H and FAD can exist in a quenched and an unquenched configuration with distinct lifetimes. To extract these lifetimes, fluorescence decays were fit to a two-component exponential decay that was re-convolved with the instrument response function:

$$I ( t ) = {α}_{1} {e}^{- \frac{t}{{τ}_{1}}} + {α}_{2} {e}^{- \frac{t}{{τ}_{2}}} + C \qquad ( 1 )$$

where I(t)=is the light intensity at time t following the laser pulse, τ₁ and τ₂ are the short (quenched) and long (unquenched) lifetimes of the fluorophore, and α₁ and α₂ are the fractional component of each lifetime. C is included to account for background light. For NAD(P)H, the short lifetime (τ₁) corresponds to unbound NAD(P)H and the long lifetime (τ₂) corresponds to protein-bound NAD(P)H 29. The opposite is true of FAD: the short and long lifetime correspond to bound FAD and unbound FAD, respectively. A mean lifetime at each pixel was also computed as the weighted average of the short and long lifetime:

$${τ}_{m} = {α}_{1} {τ}_{1} + {α}_{2} {τ}_{2} \qquad ( 2 )$$

Following extraction of the fluorescence lifetimes, images were segmented to create single-cell masks using NAD(P)H intensity images. Segmentation was carried out in CellProfiler, resulting in masks of cells, cell nuclei, and cell cytoplasm. PerCP-conjugated CD69 fluorescence images were manually segmented by a trained observer. The observer was blinded to whether PerCP-CD69 images came from the activated or unstimulated condition. The resulting masks were used to identify activated and quiescent cells in each condition based on overlap between PerCP-CD69 masks and cell masks.

Fluorescent lifetime components for each cell were calculated in R. The values of NAD(P)H τₘ, NAD(P)H τ₁, NAD(P)H τ₂, NAD(P)H α₁, FAD τₘ, FAD τ₁, FAD τ₂, and FAD α₁ were calculated for each cell by averaging across all pixels in the cell cytoplasm. Cells with low photon counts (<5000 photons), small masks that are unlikely cells (<350 pixels or 75 μm² whole cell area), and pixels with poor goodness-of-fit (χ²>1.3) were not included in this analysis. α₂ was not computed, as the sum of α₁ and α₂ is equal to 1 (100%). An additional parameter, the optical redox ratio, was computed for each cell, defined here as the NAD(P)H intensity divided by the sum of the NAD(P)H and FAD intensities. This definition of the redox ratio is bound between 0 and 1. To account for variations in intensity from day-to-day equipment and setting changes, the redox ratio of each cell was normalized to the mean redox ratio of the control group within each experiment.

### Measurement of Cytokines and Glucose/Lactate Levels in Primary Cell Media

To validate the activation of lymphocytes in each condition, cytokine levels were measured in media samples collected from both the unstimulated and activated conditions during plating (24 hours post activation for NK cells, and 72 hours post activation for B cells). IFN-γ levels were measured in NK cell media samples using the human IFN-γ DuoSet ELISA kit (R&D Systems). IL-6 levels were measured in B cell media samples using the human IL-6 DuoSet ELISA kit (R&D Systems). The ELISA assay was carried out according to the provided protocol. Plates were incubated overnight with 2 μg/mL IFN-γ or IL-6 capture antibody. The plates were then washed and blocked with a 1% bovine serum albumin solution for 1 hour. Following washing, media samples and standards were incubated on the plates for 2 hours at room temperature, followed by a 2 hour incubation with 200 ng/mL IFN-γ or 50 ng/ml IL-6 detection antibody. Finally, the plates were incubated with streptavidin-conjugated horseradish peroxidase B, then an H₂O₂-tetramethylbenzidine substrate solution. The color reaction was stopped at 20 minutes with a 4M H₂SO₄ solution, and the plates were transferred to a plate reader, where they were read at 450 nm with wavelength correction at 570 nm. Standard curves were calculated from a serial dilution of the standards using a sigmoidal four parameter logistic model. The R² of the standard curves for the IL-6 and IFN-γ ELISA experiments were 0.9993 and 0.9997, respectively.

To validate that the cells were upregulating aerobic glycolysis in the activated cell populations, commercial kits were used to measure glucose and lactate levels in media samples collected from both the unstimulated and activated conditions during plating (24 hours post activation for NK cells, and 72 hours post activation for B cells). A sample of the growth media used for the B cells and NK cells described in the “Lymphocyte activation and culture” was also evaluated as a control. The glucose and lactate assays were carried out according to the respective protocols for the Glucose Colorimetric/Fluormetric Assay Kit (BioVision) or the Lactate Colorimetric/Fluormetric Assay Kit (BioVision). 0.5 μL of each sample was added to a 96-well plate were an additional 49.5 μL of assay buffer was added, yielding a 100× dilution of the original samples. 50 μL of reaction mix (2 μL probe, 2 μL enzyme mix, and 46 μL assay buffer) was then added to each well to yield a total volume of 100 μL per well. The 96-well trays were left to incubate for 30 minutes in a dark box at room temperature (glucose assay) or 37° ° C. (lactate assay). The plates were then transferred to a plate reader where glucose or lactate levels were quantified by absorbance at OD 570. Standard curves were calculated from a serial dilution of the standards using an ordinary least squares regression model. The R² of the standard curves for the glucose and lactate assays were 0.9973 and 0.9979, respectively.

### Previous CD3+ T Cell Data

Previously published T cell data from Walsh. et. al. (2021) was used for the purposes of classifying lymphocyte subtypes in FIG. 9, FIGS. 14-17, FIG. 19, and FIG. 20. T cells were isolated from human blood and either left quiescent or stimulated with CD2/3/28 for 48 hours for activation. Activation was confirmed with a CD69-PerCP label across three donors. This prior data was collected in the same manner on the same two-photon fluorescence lifetime imaging system as the current NK cell and B cell data.

### Heatmap, UMAP, and Classification

Z-score heatmaps were constructed in R using the Complex Heatmap package 55. Clustering of groups or single cells was performed based on the OMI parameters and calculated using Ward's method. Labels for activation, lymphocyte subtype, and donor were added afterwards and were not included in cluster analysis.

Uniform Manifold Approximation and Projection (UMAP) is a non-linear dimension reduction technique that can be used to visualize high-dimensional data. UMAP projections were made in Python using scikit-learn, UMAP, and Holoviews. Unless otherwise noted, each UMAP is a two-dimensional visualization of 9 variables (normalized optical redox ratio; NAD(P)H τₘ, τ₁, τ₂, α₁; FAD τₘ, τ₁, τ₂, α₁). The UMAP projection was computed using Euclidean distance. The nearest neighbors parameter was set to 15 and the minimum distance was set to 0.4 unless otherwise noted.

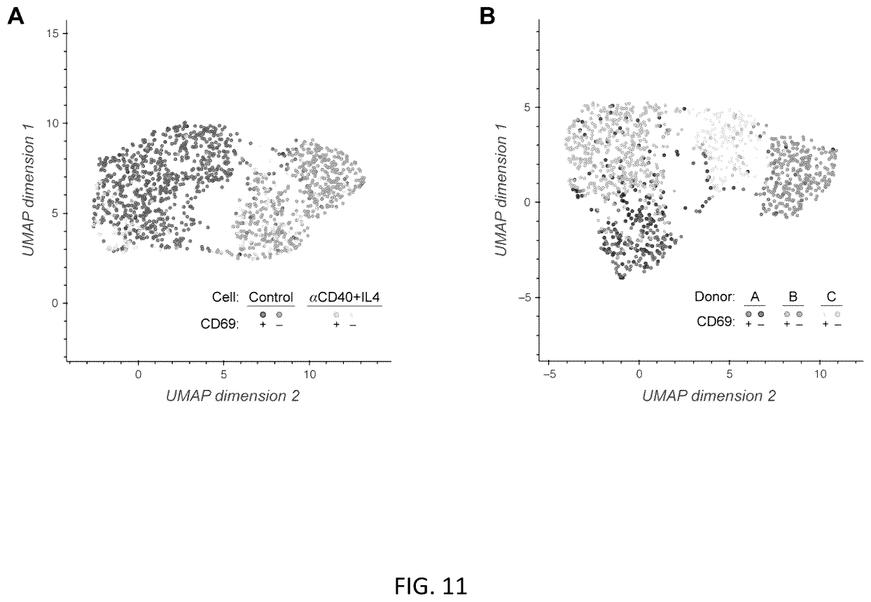

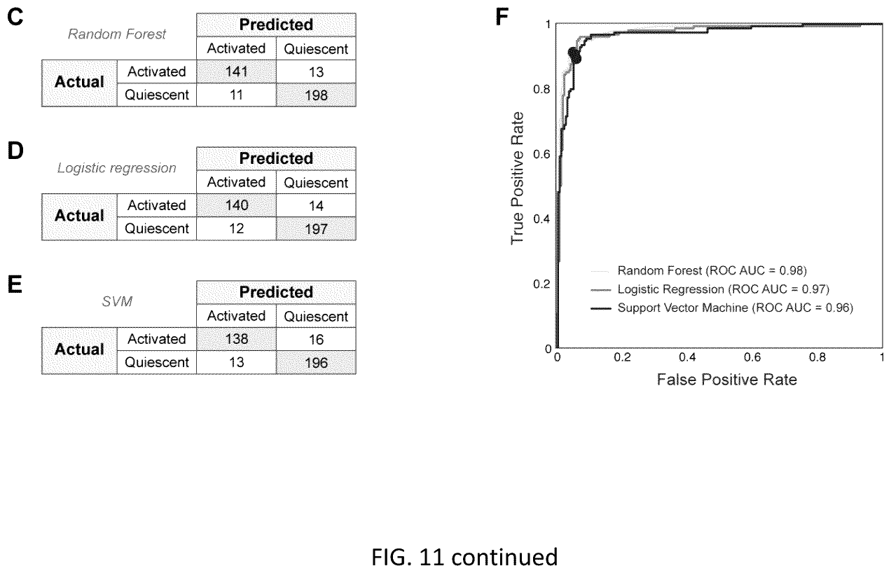

Random forest classification methods were trained in Python using scikit-learn to classify activation and/or lymphocyte subtype in the NK cell OMI parameters, the B cell OMI parameters, or combined OMI parameters from NK cells, B cells, and previously published T cell data 47. The classifier was trained on a random selection of 70% of the input data and tested on the remaining 30% for B cell or NK cell classifiers alone (i.e., FIGS. 6, 8, and FIGS. 11, 13). For the classifiers using a combined lymphocyte dataset of B, NK, and T cells (i.e., FIG. 9 and FIGS. 14-17), the classifier was trained on a random selection of 50% of the input data and tested on the remaining 50%. Multiple metrics were used to evaluate the robustness of the classifier, including the receiver operating characteristic (ROC) curve, accuracy, precision, and recall. Classifiers were trained and tested on different random sets of the data to check for consistency in these metrics. Equal cost was given to a misclassified cell regardless of category (i.e., misclassification was not weighted by sample size).

Phasor-based classification was performed using the NAD(P)H and FAD phasor coordinates (G,S) at the laser repetition frequency (80 MHZ) and its second harmonic (160 MHz) as features. The phasor coordinates were averaged pixel-wise over each cell mask using pixel intensities as weights to calculate cell-level phasor coordinates. Logistic regression classifiers with a log it link function and random forest classifiers with 100 decision trees were used, and the classifiers were trained on a random selection of 50% of the input data and tested on the remaining 50%. Again, equal cost was given to a misclassified cell regardless of category (i.e., misclassification was not weighted by sample size). Both the phasor and the fit analysis pipelines use the same raw FLIM data and cell masks to calculate cell-level phasor coordinates and fit parameters, respectively. However, the exclusion criteria for the two pipelines are not the same, which results in different final number of cells included in the phasor-based and fit-based classifiers. For example, the phasor pipeline removes low-count (with fewer than 5000 photons) or small (with fewer than 50 pixels) cells, while the fit analysis also removes cells based on the goodness of the bi-exponential fit (χ²>1.3).

### Statistical Analysis

Statistical analysis was performed using the statannotations package v0.5.0 in Python. Differences between groups were tested using Kruskal-Wallis with post-hoc comparisons test for multiple group comparisons, or a two-tailed unpaired T-test for comparisons of pairs of data.

References—each of the following documents is incorporated herein in its entirety by reference for all purposes:
1. Omman, R. A., and Kini, A. R. (2020). 9—Leukocyte development, kinetics, and functions. In Rodak's Hematology (Sixth Edition), E. M. Keohane, C. N. Otto, and J. M. Walenga, eds. (Elsevier), pp. 117-135. 10.1016/B978-0-323-53045-3.00018-0.
2. Weber, E. W., Maus, M. V., and Mackall, C. L. (2020). The Emerging Landscape of Immune Cell Therapies. Cell 181, 46-62. 10.1016/j.cell.2020.03.001.
3. Vivier, E., Tomasello, E., Baratin, M., Walzer, T., and Ugolini, S. (2008). Functions of natural killer cells. Nat. Immunol. 9, 503-510. 10.1038/ni1582.
4. Hu, W., Wang, G., Huang, D., Sui, M., and Xu, Y. (2019). Cancer Immunotherapy Based on Natural Killer Cells: Current Progress and New Opportunities. Front. Immunol. 10, 1205. 10.3389/fimmu.2019.01205.
5. Romee, R., Rosario, M., Berrien-Elliott, M. M., Wagner, J. A., Jewell, B. A., Schappe, T., Leong, J. W., Abdel-Latif, S., Schneider, S. E., and Willey, S. (2016). Cytokine-induced memory-like natural killer cells exhibit enhanced responses against myeloid leukemia. Sci. Transl. Med. 8, 357ra123-357ra123.
6. Wennhold, K., Shimabukuro-Vornhagen, A., and von Bergwelt-Baildon, M. (2019). B Cell-Based Cancer Immunotherapy. Transfus. Med. Hemotherapy 46, 36-46. 10.1159/000496166.
7. Bonilla, F. A., and Oettgen, H. C. (2010). Adaptive immunity. J. Allergy Clin. Immunol. 125, S33-S40. 10.1016/j.jaci.2009.09.017.
8. Hoffman, W., Lakkis, F. G., and Chalasani, G. (2016). B Cells, Antibodies, and More. Clin. J. Am. Soc. Nephrol. CJASN 11, 137-154. 10.2215/CJN.09430915.
9. Hung, K. L., Meitlis, I., Hale, M., Chen, C.-Y., Singh, S., Jackson, S. W., Miao, C. H., Khan, I. F., Rawlings, D. J., and James, R. G. (2018). Engineering Protein-Secreting Plasma Cells by Homology-Directed Repair in Primary Human B Cells. Mol. Ther. 26, 456-467. 10.1016/j.ymthe.2017.11.012.
10. Mpande, C. A. M., Rozot, V., Mosito, B., Musvosvi, M., Dintwe, O. B., Bilek, N., Hatherill, M., Scriba, T. J., and Nemes, E. (2021). Immune profiling of Mycobacterium tuberculosis-specific T cells in recent and remote infection. EBioMedicine 64, 103233. 10.1016/j.ebiom.2021.103233.
11. Vendrame, E., McKechnie, J. L., Ranganath, T., Zhao, N. Q., Rustagi, A., Vergara, R., Ivison, G. T., Kronstad, L. M., Simpson, L. J., and Blish, C. A. (2020). Profiling of the Human Natural Killer Cell Receptor-Ligand Repertoire. J. Vis. Exp. JoVE, 10.3791/61912. 10.3791/61912.
12. Cho, Y.-H., Choi, M. G., Kim, D. H., Choi, Y. J., Kim, S. Y., Sung, K. J., Lee, J. C., Kim, S.-Y., Rho, J. K., and Choi, C.-M. (2020). Natural Killer Cells as a Potential Biomarker for Predicting Immunotherapy Efficacy in Patients with Non-Small Cell Lung Cancer. Target. Oncol. 15, 241-247. 10.1007/s11523-020-00712-2.
13. Pasero, C., Gravis, G., Granjeaud, S., Guerin, M., Thomassin-Piana, J., Rocchi, P., Salem, N., Walz, J., Moretta, A., and Olive, D. (2015). Highly effective NK cells are associated with good prognosis in patients with metastatic prostate cancer. Oncotarget 6, 14360-14373. 10.18632/oncotarget.3965.
14. van der Leun, A. M., Thommen, D. S., and Schumacher, T. N. (2020). CD8+ T cell states in human cancer: insights from single-cell analysis. Nat. Rev. Cancer 20, 218-232. 10.1038/s41568-019-0235-4.
15. Tsioris, K., Gupta, N. T., Ogunniyi, A. O., Zimnisky, R. M., Qian, F., Yao, Y., Wang, X., Stern, J. N. H., Chari, R., Briggs, A. W., et al. (2015). Neutralizing antibodies against West Nile virus identified directly from human B cells by single-cell analysis and next generation sequencing. Integr. Biol. 7, 1587-1597. 10.1039/c5ib00169b.
16. Rezvani, K. (2019). Adoptive cell therapy using engineered natural killer cells. Bone Marrow Transplant. 54, 785-788. 10.1038/s41409-019-0601-6.
17. Boonyaratanakornkit, J., and Taylor, J. J. (2019). Techniques to Study Antigen-Specific B Cell Responses. Front. Immunol. 10.
18. Chen, G., Ning, B., and Shi, T. (2019). Single-Cell RNA-Seq Technologies and Related Computational Data Analysis. Front. Genet. 10.
19. Marcu, L. (2012). Fluorescence lifetime techniques in medical applications. Ann. Biomed. Eng. 40, 304-331. 10.1007/s10439-011-0495-y.
20. Skala, M. C., Riching, K. M., Gendron-Fitzpatrick, A., Eickhoff, J., Eliceiri, K. W., White, J. G., and Ramanujam, N. (2007). In vivo multiphoton microscopy of NADH and FAD redox states, fluorescence lifetimes, and cellular morphology in precancerous epithelia. Proc. Natl. Acad. Sci. 104, 19494-19499. 10.1073/pnas.0708425104.
21. Awasthi, K., Moriya, D., Nakabayashi, T., Li, L., and Ohta, N. (2016). Sensitive detection of intracellular environment of normal and cancer cells by autofluorescence lifetime imaging. J. Photochem. Photobiol. B 165, 256-265. 10.1016/j.jphotobiol.2016.10.023.
22. Schaefer, P. M., Kalinina, S., Rueck, A., von Arnim, C. A. F., and von Einem, B. (2019). NADH Autofluorescence-A Marker on its Way to Boost Bioenergetic Research. Cytometry A 95, 34-46. 10.1002/cyto.a.23597.
23. Georgakoudi, I., and Quinn, K. P. (2012). Optical Imaging Using Endogenous Contrast to Assess Metabolic State. Annu. Rev. Biomed. Eng. 14, 351-367. 10.1146/annurev-bioeng-071811-150108.
24. Nakashima, N., Yoshihara, K., Tanaka, F., and Yagi, K. (1980). Picosecond fluorescence lifetime of the coenzyme of D-amino acid oxidase. J. Biol. Chem. 255, 5261-5263. 10.1016/S0021-9258(19)70779-0.
25. Huang, S., Heikal, A. A., and Webb, W. W. (2002). Two-Photon Fluorescence Spectroscopy and Microscopy of NAD(P)H and Flavoprotein. Biophys. J. 82, 2811-2825. 10.1016/S0006-3495(02)75621-X.
26. Chance, B., Schoener, B., Oshino, R., Itshak, F., and Nakase, Y. (1979). Oxidation-reduction ratio studies of mitochondria in freeze-trapped samples. NADH and flavoprotein fluorescence signals. J. Biol. Chem. 254, 4764-4771. 10.1016/S0021-9258(17)30079-0.
27. Ostrander, J. H., McMahon, C. M., Lem, S., Millon, S. R., Brown, J. Q., Seewaldt, V. L., and Ramanujam, N. (2010). Optical Redox Ratio Differentiates Breast Cancer Cell Lines Based on Estrogen Receptor Status. Cancer Res. 70, 4759-4766. 10.1158/0008-5472.CAN-09-2572.
28. Suhling, K., French, P. M. W., and Phillips, D. (2005). Time-resolved fluorescence microscopy. Photochem. Photobiol. Sci. 4, 13-22. 10.1039/B412924P.
29. Lakowicz, J. R., Szmacinski, H., Nowaczyk, K., and Johnson, M. L. (1992). Fluorescence lifetime imaging of free and protein-bound NADH. Proc. Natl. Acad. Sci. U.S.A 89, 1271-1275.
30. Sharick, J. T., Favreau, P. F., Gillette, A. A., Sdao, S. M., Merrins, M. J., and Skala, M. C. (2018). Protein-bound NAD(P)H Lifetime is Sensitive to Multiple Fates of Glucose Carbon. Sci. Rep. 8, 5456. 10.1038/s41598-018-23691-x.
31. Walsh, A. J., and Skala, M. C. (2015). Optical metabolic imaging quantifies heterogeneous cell populations. Biomed. Opt. Express 6, 559-573. 10.1364/BOE.6.000559.
32. Cong, J. (2020). Metabolism of Natural Killer Cells and Other Innate Lymphoid Cells. Front. Immunol. 11, 1989. 10.3389/fimmu.2020.01989.
33. Gardiner, C. M., and Finlay, D. K. (2017). What Fuels Natural Killers? Metabolism and NK Cell Responses. Front. Immunol. 8, 367. 10.3389/fimmu.2017.00367.
34. O'Brien, K. L., and Finlay, D. K. (2019). Immunometabolism and natural killer cell responses. Nat. Rev. Immunol. 19, 282-290. 10.1038/s41577-019-0139-2.
35. Ripperger, T. J., and Bhattacharya, D. (2021). Transcriptional and Metabolic Control of Memory B Cells and Plasma Cells. Annu. Rev. Immunol. 39, 345-368. 10.1146/annurev-immunol-093019-125603.
36. Chapman, N. M., and Chi, H. (2022). Metabolic adaptation of lymphocytes in immunity and disease. Immunity 55, 14-30. 10.1016/j.immuni.2021.12.012.
37. Donnelly, R. P., Loftus, R. M., Keating, S. E., Liou, K. T., Biron, C. A., Gardiner, C. M., and Finlay, D. K. (2014). mTORC1-Dependent Metabolic Reprogramming Is a Prerequisite for NK Cell Effector Function. J. Immunol. 193, 4477-4484. 10.4049/jimmunol. 1401558.
38. Keating, S. E., Zaiatz-Bittencourt, V., Loftus, R. M., Keane, C., Brennan, K., Finlay, D. K., and Gardiner, C. M. (2016). Metabolic Reprogramming Supports IFN-γ Production by CD56bright NK Cells. J. Immunol. 196, 2552-2560. 10.4049/jimmunol. 1501783.
39. Khalsa, J. K., Chawla, A. S., Prabhu, S. B., Vats, M., Dhar, A., Dev, G., Das, N., Mukherjee, S., Tanwar, S., Banerjee, H., et al. (2019). Functionally significant metabolic differences between B and T lymphocyte lineages. Immunology 158, 104-120. 10.1111/imm.13098.
40. Assmann, N., O'Brien, K. L., Donnelly, R. P., Dyck, L., Zaiatz-Bittencourt, V., Loftus, R. M., Heinrich, P., Oefner, P. J., Lynch, L., Gardiner, C. M., et al. (2017). Srebp-controlled glucose metabolism is essential for NK cell functional responses. Nat. Immunol. 18, 1197-1206. 10.1038/ni.3838.
41. Poznanski, S. M., Barra, N. G., Ashkar, A. A., and Schertzer, J. D. (2018). Immunometabolism of T cells and NK cells: metabolic control of effector and regulatory function. Inflamm. Res. 67, 813-828. 10.1007/s00011-018-1174-3.
42. Heaster, T. M., Humayun, M., Yu, J., Beebe, D. J., and Skala, M. C. (2020). Autofluorescence Imaging of 3D Tumor-Macrophage Microscale Cultures Resolves Spatial and Temporal Dynamics of Macrophage Metabolism. Cancer Res. 80, 5408-5423. 10.1158/0008-5472.CAN-20-0831.
43. Yakimov, B. P., Gogoleva, M. A., Semenov, A. N., Rodionov, S. A., Novoselova, M. V., Gayer, A. V., Kovalev, A. V., Bernakevich, A. I., Fadeev, V. V., Armaganov, A. G., et al. (2019). Label-free characterization of white blood cells using fluorescence lifetime imaging and flow-cytometry: molecular heterogeneity and erythrophagocytosis [Invited]. Biomed. Opt. Express 10, 4220-4236. 10.1364/BOE.10.004220.
44. Miskolci, V., Tweed, K. E., Lasarev, M. R., Britt, E. C., Walsh, A. J., Zimmerman, L. J., McDougal, C. E., Cronan, M. R., Fan, J., Sauer, J.-D., et al. (2022). In vivo fluorescence lifetime imaging of macrophage intracellular metabolism during wound responses in zebrafish. eLife 11, e66080. 10.7554/eLife.66080.
45. Lemire, S., Thoma, O.-M., Kreiss, L., Volkl, S., Friedrich, O., Neurath, M. F., Schürmann, S., and Waldner, M. J. (2022). Natural NADH and FAD Autofluorescence as Label-Free Biomarkers for Discriminating Subtypes and Functional States of Immune Cells. Int. J. Mol. Sci. 23, 2338. 10.3390/ijms23042338.
46. Charles A Janeway, J., Travers, P., Walport, M., and Shlomchik, M. J. (2001). B-cell activation by armed helper T cells. Immunobiol. Immune Syst. Health Dis. 5th Ed.
47. Walsh, A. J., Mueller, K., Tweed, K., Jones, I., Walsh, C. M., Piscopo, N., Niemi, N. M., Pagliarini, D. J., Saha, K., and Skala, M. C. (2021). Classification of T-cell activation via autofluorescence lifetime imaging. Nat. Biomed. Eng. 5, 77-88. 10.1038/s41551-020-0592-z.
48. Jones, R. G., and Thompson, C. B. (2007). Revving the Engine: Signal Transduction Fuels T Cell Activation. Immunity 27, 173-178. 10.1016/j.immuni.2007.07.008.
49. Walsh, A. J., Cook, R. S., Manning, H. C., Hicks, D. J., Lafontant, A., Arteaga, C. L., and Skala, M. C. (2013). Optical Metabolic Imaging Identifies Glycolytic Levels, Subtypes, and Early-Treatment Response in Breast Cancer. Cancer Res. 73, 6164-6174. 10.1158/0008-5472.CAN-13-0527.
50. Liu, Z., Pouli, D., Alonzo, C. A., Varone, A., Karaliota, S., Quinn, K. P., Münger, K., Karalis, K. P., and Georgakoudi, I. (2018). Mapping metabolic changes by noninvasive, multiparametric, high-resolution imaging using endogenous contrast. Sci. Adv. 4, eaap9302. 10.1126/sciadv.aap9302.
51. Cooper, M. A., Elliott, J. M., Keyel, P. A., Yang, L., Carrero, J. A., and Yokoyama, W. M. (2009). Cytokine-induced memory-like natural killer cells. Proc. Natl. Acad. Sci. 106, 1915-1919. 10.1073/pnas.0813192106.
52. Ni, J., Miller, M., Stojanovic, A., Garbi, N., and Cerwenka, A. (2012). Sustained effector function of IL-12/15/18-preactivated NK cells against established tumors. J. Exp. Med. 209, 2351-2365. 10.1084/jem.20120944.
53. Van Belle, K., Herman, J., Boon, L., Waer, M., Sprangers, B., and Louat, T. (2016). Comparative In Vitro Immune Stimulation Analysis of Primary Human B Cells and B Cell Lines. J. Immunol. Res. 2016, 5281823. 10.1155/2016/5281823.
54. Duddy, M. E., Alter, A., and Bar-Or, A. (2004). Distinct Profiles of Human B Cell Effector Cytokines: A Role in Immune Regulation? J. Immunol. 172, 3422-3427. 10.4049/jimmunol.172.6.3422.
55. Gu, Z., Eils, R., and Schlesner, M. (2016). Complex heatmaps reveal patterns and correlations in multidimensional genomic data. Bioinformatics 32, 2847-2849. 10.1093/bioinformatics/btw313.

We claim:

1. A lymphocyte activation and/or identification status sensing device comprising:
a cell analysis observation zone adapted to receive a lymphocyte and to present the lymphocyte for individual autofluorescence interrogation;
an autofluorescence spectrometer configured to acquire an autofluorescence data set for the lymphocyte located in the cell analysis observation zone, the autofluorescence spectrometer comprising a light source, a photon-counting detector, and photon-counting electronics;
a processor in electronic communication with the autofluorescence spectrometer; and
a non-transitory computer-readable medium accessible to the processor and having stored thereon instructions that, when executed by the processor, cause the processor to:
a) receive the autofluorescence data set; and
b) identify a current activation and/or identification status of the lymphocyte based on a current activation and/or identification prediction, wherein the current activation and/or identification prediction is computed using at least a portion of the autofluorescence data set,
wherein the current activation and/or identification prediction is:
i) a six-class classification that predicts that the lymphocyte is an activated T cell, a quiescent T cell, an activated B cell, a quiescent B cell, an activated NK cell, or a quiescent NK cell, wherein the six-class classification is computed using at least three six-class metabolic endpoints as an input, wherein the at least three six-class metabolic endpoints include reduced nicotinamide adenine dinucleotide and/or reduced nicotinamide dinucleotide phosphate (NAD(P)H) shortest fluorescence amplitude component (α1), NAD(P)H shortest fluorescence lifetime component (τ1), and NAD(P)H mean fluorescence lifetime (τm);
ii) a lymphocyte identification prediction that predicts that the lymphocyte is a T cell, a B cell, or a NK cell, wherein the lymphocyte identification prediction is computed using at least two metabolic endpoints as an input, wherein the at least two lymphocyte identification metabolic endpoints include flavin adenine dinucleotide (FAD) τm, FAD τ1, or NAD(P)H τm;
iii) a lymphocyte activation prediction that predicts that the lymphocyte is an activated lymphocyte or a quiescent lymphocyte, wherein the lymphocyte activation prediction is computed using at least two lymphocyte activation metabolic endpoints as an input, wherein the at least two lymphocyte activation metabolic endpoints include NAD(P)H α1 and one of an optical redox ratio, NAD(P)H τ1, or NAD(P)H τm;
iv) a B cell activation prediction that predicts that the lymphocyte is an activated B cell or a quiescent B cell, wherein the B cell activation prediction is computed using at least two B cell activation metabolic endpoints as an input, wherein the at least two B cell metabolic endpoints include NAD(P)H α1 and NAD(P)H τm; or
v) a NK cell activation prediction that predicts that the lymphocyte is an activated NK cell or a quiescent NK cell, wherein the NK cell activation prediction is computed using at least two NK cell activation metabolic endpoints as an input, wherein the at least two NK activation metabolic endpoints include either: NAD(P)H α1 and an optical redox ratio; or NAD(P)H α1, NAD(P)H τm, and NAD(P)H second shortest lifetime (τ2), and NAD(P)H τ1.

2. The lymphocyte activation and/or identification status sensing device of claim 1, the device further comprising a cell analysis platform adapted to receive a cell culture containing the B cell, the cell analysis platform adapted to position the cell culture containing the B cell in the observation zone.

3. The lymphocyte activation and/or identification status sensing device of claim 1, the device further comprising a cell analysis pathway comprising: (i) an inlet; (ii) the observation zone coupled to the inlet downstream of the inlet, the observation zone configured to present the lymphocyte for individual autofluorescence interrogation; and (iii) an outlet coupled to the observation zone downstream of the observation zone.

4. The lymphocyte activation and/or identification status sensing device of claim 3, wherein the cell analysis pathway comprises a microfluidic pathway or a nanofluidic pathway.

5. The lymphocyte activation and/or identification status sensing device of claim 3, the lymphocyte activation and/or identification status sensing device further comprising a flow regulator coupled to the inlet.

6. The lymphocyte activation and/or identification status sensing device of claim 5, wherein the flow regulator is configured to provide flow of cells through the observation zone at a rate that allows the autofluorescence spectrometer to acquire the autofluorescence data set for the lymphocyte when it is positioned in the observation zone.

7. The lymphocyte activation and/or identification status sensing device of claim 3, wherein the cell analysis pathway does not include a fluorescent label for binding to the lymphocyte.

8. The lymphocyte activation and/or identification status sensing device of claim 3, wherein the cell analysis pathway does not include an immobilization agent for binding and immobilizing the lymphocyte.

9. The lymphocyte activation and/or identification status sensing device claim 3, the lymphocyte activation and/or identification status sensing device further comprising a cell sorter having a sorter inlet and at least two sorter outlets, the cell sorter coupled to the cell analysis pathway via the outlet downstream of the observation zone, the cell sorter configured to selectively direct a cell from the sorter inlet to one of the at least two sorter outlets based on a sort signal, the processor in electronic communication with the cell sorter, and the instructions, when executed by the processor, further cause the processor to provide the sort signal to the cell sorter based on the current activation prediction.

10. The lymphocyte activation and/or identification status sensing device of claim 3, the lymphocyte activation and/or identification status sensing device further comprising a cell picker.

11. The lymphocyte activation and/or identification status sensing device of claim 10, wherein the cell picker automatically picks a cell based on the current activation and/or identification prediction.

12. The lymphocyte activation and/or identification status sensing device of claim 3, the autofluorescence spectrometer comprising a detector-side filter configured to transmit fluorescence signals of interest.

13. The lymphocyte activation and/or identification status sensing device of claim 3, the lymphocyte activation and/or identification status sensing device further comprising at least one of a cell size measurement tool configured to measure cell size and to communicate the cell size to the processor and a cell imager configured to acquire an image of a cell positioned within the observation zone and to communicate the image to the processor.

14. The lymphocyte activation and/or identification status sensing device of claim 3, wherein the autofluorescence spectrometer is adapted to measure autofluorescence of the lymphocyte without requiring the use of a fluorescent label.

15. The lymphocyte activation and/or identification status sensing device of claim 3, wherein the instructions, when executed by the processor, further cause the processor to generate a report including the current activation and/or identification prediction for lymphocytes analyzed by the device.

16. A method of six-class classification of lymphocyte activation and identification status, the method comprising:
a) receiving a population of lymphocytes having unknown activation and identification status;
b) acquiring an autofluorescence data set for each lymphocyte of the population of lymphocytes, each autofluorescence data set including autofluorescence lifetime information; and
either:
c1) physically isolating the population of lymphocytes into at least six separate portions based on a six-class classification that predicts that each lymphocyte is an activated T cell, a quiescent T cell, an activated B cell, a quiescent B cell, an activated NK cell, or a quiescent NK cell;
c2) generating a report including the six-class classification, the report optionally identifying a proportion of the population of lymphocytes having a given six-class classification,
wherein the six-class classification is computed using at least a portion of the autofluorescence data set, wherein the six-class classification is computed using at least three six-class metabolic endpoints as an input,
wherein the at least three six-class metabolic endpoints include reduced nicotinamide adenine dinucleotide and/or reduced nicotinamide dinucleotide phosphate (NAD(P)H) shortest fluorescence amplitude component (α1), NAD(P)H shortest fluorescence lifetime component (τ1), and NAD(P)H mean fluorescence lifetime (τm).

17. A method of administering activated lymphocytes to a subject in need thereof, the method comprising:
d) the method of claim 16, wherein the method comprising step c1); and
e) introducing one or more of the six separate portions to a subject, wherein the one or more of the six separate portions is predicted to be activated lymphocytes.

18. The method of claim 17, wherein the one or more of the six separate portions is modified prior to step e).

19. A method of administering activated lymphocytes to a subject in need thereof, the method comprising:
d) the method of claim 16, wherein the method comprises step c2); and
e) in response to the proportion of the population of lymphocytes having a given six-class classification exceeding a predetermined threshold, introducing the population of lymphocytes to the subject.

20. The method of claim 19, wherein the population of lymphocytes is modified prior to step e).
